(19) United States
(12) Patent Application Publication
(10) Pub. No.: US 2002/0165178 A1
(43) Pub. Date: Nov. 7, 2002

(54) Immunostimulatory nucleic acids for the treatment of anemia, thrombocytopenia, and neutropenia

(76) Inventors: Christian Schetter, Hilden (DE); Robert L. Bratzler, Concord, MA (US); Deanna M. Petersen, Newton, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/895,007

(22) Filed: Jun. 28, 2001

Related U.S. Application Data
(60) Provisional application No. 60/214,368, filed on Jun. 28, 2000.

(51) Int. Cl. A61K 48/00; C07H 21/04

(52) U.S. Cl. 514/44; 536/23.2

(57) ABSTRACT

The invention involves administration of an immunostimulatory nucleic acid alone or in combination with an anemia, thrombocytopenia, or neutropenia medicament for the treatment or prevention of anemia, thrombocytopenia, and neutropenia in subjects. The agents in combination are administered in synergistic amounts or in various dosages or at various time schedules. The invention also relates to kits and compositions concerning the combination of immunostimulatory nucleic acids and anemia, thrombocytopenia, or neutropenia drugs.

### PRIORITY

This application claims benefit of U.S. Provisional Application No. 60/214,368, filed Jun. 28, 2000.

### FIELD OF THE INVENTION

The present invention relates to immunostimulatory nucleic acids, compositions thereof and methods of using the immunostimulatory nucleic acids in the treatment of anemia, thrombocytopenia, and neutropenia.

### BACKGROUND OF THE INVENTION

Hematopoiesis, the formation of blood cells, represents a complex physiologic phenomenon whereby blood cells of various lineages arise from common progenitor cells called hematopoietic stem cells. Hematopoietic development is regulated by colony-stimulating factors (CSFs), which promote colony formation and proliferation of cells of various lineages, and by potentiators, which potentiate maturation or differentiation. Many of the factors involved in hematopoiesis affect more than a single lineage. For example, erythropoietin (EPO) stimulates both erythrocyte and platelet production. Similarly, some factors can be classified both as CSF and as potentiator. For example, thrombopoietin (TPO) was reported to possess both megakaryocyte-CSF (Meg-CSF) and megakaryocyte potentiator (Meg-Pot) activity in the development of megakaryocytes in vivo. In addition, many factors are involved in the development of any given cell lineage. Thus Meg-CSFs reportedly include interleukin-3 (IL-3), granulocyte-macrophage colony-stimulating factor (GM-CSF) and stem cell factor, and Meg-Pots reportedly include IL-6, IL-7, IL-11, erythropoietin (EPO) and leukemia inhibitory factor (LIF).

Hematopoiesis is necessary under normal and stress conditions. Under normal conditions, senescent mature cells are continuously removed and replaced with newly generated cells. Under stress conditions, there may be an increased rate at which blood cells are destroyed or lost, or there may be a compromised capacity to replenish cells undergoing normal senescent attrition, resulting in depletion of erythrocytes (anemia), platelets (thrombocytopenia), and/or leukocytes (leukopenia).

Radiation and chemotherapeutic treatment frequently produce severe reversible neutropenia, thrombocytopenia, and anemia. This effect comes about as the result of depletion of hematopoietic precursors and of the cells responsible for producing the required CSFs and hematopoietic potentiators. The depletion of hematopoietic precursors in the bone marrow associated with chemotherapy and irradiation sometimes results in life-threatening hemorrhagic and infectious complications. Severe suppression of hematopoiesis is a major factor in limiting chemotherapy use and dose escalation. Replacement of depleted blood cell types by transfusion is not always practical or desirable. Such transfusion often affords only temporary improvement, is expensive, and is associated with risks of infection, fluid overload, and immune-mediated adverse reactions. Thus there has been intense interest in developing methods of using hematopoietic CSFs and potentiators to treat neutropenia, thrombocytopenia, and anemia.

In recent years three recombinant human hematopoietic growth factors became available for clinical use: EPO for the treatment of anemia, and granulocyte colony-stimulating factor (G-CSF) and GM-CSF for neutropenia. While these factors have proven to be generally safe and effective, they are expensive. Nevertheless, other hematopoietic growth factors and cytokines, including TPO and IL-3, IL-6, and IL-11, are under development and/or study as potential hematopoietic agents.

Certain cytokines and growth factors, such as IL-3, IL-6, IL-11, IL-12, G-CSF, and GM-CSF are generally regarded as inducers of hematopoiesis, while others, particularly interferon gamma (IFN-γ) and tumor necrosis factor alpha (TNF-α), are generally regarded as suppressors of hematopoiesis. These broad categories of growth factors and cytokines in terms of their hematopoietic effects do not adhere to the otherwise useful categories of cytokines in terms of their Th1 and Th2 character in generating an immune response.

Hematopoietic growth factors and cytokines characteristically exert multiple biologic effects. For example TPO, in addition to inducing megakaryocytes, induces red blood cell production as well. G-CSF has been shown to promote recovery of not only white blood cells but also red blood cells and platelets following sublethal radiation. Tanikawa et al. (1989) Exp Hematol 17:883-8. Similarly, IL-12 has been found to enhance erythropoiesis, granulopoiesis, and megakaryocytopoiesis in mice following sublethal irradiation. Wang et al. (1997) Chung Hua I Hsueh Tsa Chih 77:216-9. The complexity of these factors' multiple biologic effects extends to specific circumstances of site of action, dose dependence, and competing effects. Such considerations make it difficult to predict what effect to expect upon the administration of individual cytokines, even those which are generally classified as inducers of hematopoiesis.

For example, subsequent studies of IL-12 in non-irradiated mice revealed that IL-12 suppresses hematopoiesis in the bone marrow but enhances hematopoiesis in the spleen. Tare et al. (1995) J Interferon Cytokine Res 15:377-83; Jackson et al. (1995) Blood 85:2371-6. Previous studies in mice have shown that IL-6 administered to mice at 500-1000 μg/kg/day accelerated post-irradiation hematopoietic regeneration. Pojda et al. (1992) Exp Hematol 20:862-7; Laterveer et al. (1993) Exp Hematol 21:1621-7. The observed reconstitution of platelets beginning at day 12 after irradiation in response to high dose IL-6 was also observed in splenectomized mice. IL-6 given at lower doses in these same models appeared to suppress hematopoietic regeneration.

A number of agents for treatment of thrombocytopenia are currently in investigational studies. These include recombinant TPO, pegylated recombinant human megakaryocyte growth and development factor (PEG-rHuMGDF), lisofylline (Clark et al. (1996) Cancer Res 56:105-12), recombinant IL-1, recombinant IL-3, recombinant IL-6, recombinant IL-11, and recombinant G-CSF, among others. Certain of these agents, TPO and PEG-rHuMGDF among them, have been shown to promote multi-lineage hematopoiesis following myelosuppressive treatment. Miyazaki and Kato (1999) Int J Hematol 70:216-25; Ulich et al. (1999) Exp Hematol 27:1776-81; Clarke et al. (1996) Cancer Res 56:105-12; Grossmann et al. (1996) Exp Hematol 24:1238-46; Takatsuki et al. (1990) Cancer Res 50:2885-90; Leonard et al. (1994) Blood 83:1499-506. Certain of these experimental treatments for thrombocytopenia appear to be limited by lack of efficacy, toxicity, or both. For example, treatment with recombinant IL-3 has been disappointing in terms of supporting platelet and granulocyte production and complicated by unacceptable toxicity. Similarly, treatment with recombinant IL-6 has been disappointing in terms of supporting platelet production and is also limited by toxicity.

In the last two decades investigators working in the fields of cancer immunotherapy and antisense independently came to appreciate that certain nucleic acids can activate cells of the immune system. Yamamoto and colleagues, studying Bacille Calmette-Guérin (BCG)-mediated tumor resistance in mice, originally discovered that a fraction derived from BCG not only had an anti-tumor effect in vivo, but also directly augmented NK cell activity and induced secretion of IFN from peripheral blood lymphocytes in vitro. Tokunaga T et al. (1984) J Natl Cancer Inst 72:955-962; Yamamoto S et al. (1988) Jpn J Cancer Res 79:866-873; Mashiba H et al. (1988) Jpn J Med Sci Biol 41:197-202. Further studies by Yamamoto revealed the active component to be DNA, specifically 45-mers characterized by certain palindromic sequences. Synthetic 45-mer oligodeoxynucleotides (ODNs) derived from BCG cDNA sequences containing these palindromes also activated NK cells and induced secretion of IFN from PBL in vitro. Tokunaga T et al. (1992) Microbiol Immunol 36:55-66.

Subsequently, Krieg et al. formulated a framework for understanding the pattern recognition of bacterial or synthetic DNA. Krieg A M et al. (1995) Nature 374:546-549. Using sequence-specific ODN-mediated B cell mitogenicity as an assay, they discovered that certain ODN containing unmethylated CpG dinucleotides (CpG-ODN), specifically sequences containing the motif 5′-Pu-Pu-CpG-Pyr-Pyr-3′, induced murine B lymphocytes to proliferate and to secrete immunoglobulin in vitro and in vivo. Further work also revealed that the backbone of the ODN influenced the immunogenicity of ODNs. Thus for example, phosphorothiate backbone ODNs were found to be more stimulatory than phosphodiester ODNs.

The immunostimulatory effects of CpG-ODN further include the activation of professional antigen-presenting cells in vitro to secrete large amounts of IL-1, IL-3, IL-6, IL-12, GM-CSF, and TNF-α. On balance, CpG-ODN characteristically skews an immune response strongly toward a Th1-type phenotype and away from a Th2-type phenotype, i.e., toward an immune response dominated by IFN-γ and IL-12. This has been applied to advantage in its use as a T-cell adjuvant and as a treatment for Th2-mediated allergy and asthma.

According to the prior art, strong Th1 responses as characterized by IFN-γ release, and as expected to occur in response to administration of CpG DNA, may be inhibitory for hematopoiesis events. IL-12 has been shown to be released in response to CpG-ODN and is an inducer of IFN-γ, and several studies have noted the in vitro inhibition of colony forming units (CFUs) by IFN-γ. For example, normal splenic architecture was completely effaced in mycobacteria-infected mice genetically deficient for IFN-γ by expansion of macrophages, granulocytes, and extramedullary hematopoietic tissue. Murray P J et al. (1998) Blood 91:2914-2924. These features coincided with splenomegaly, an increase in splenic myeloid colony-forming activity, and marked granulocytosis in the peripheral blood. Systemic levels of cytokines were elevated, particularly IL-6 and G-CSF.

The findings of Murray et al. notwithstanding, Sparwasser et al. recently described their observation that CpG-ODN induced extramedullary hemopoiesis in mice. Sparwasser T et al. (1999) J Immunol 162:2368-2374. They found that a single intraperitoneal administration of CpG-ODN, in a sequence- and dose-dependent manner, transiently induced splenomegaly with increased numbers of non-B, non-T spleen cells. The splenomegaly was associated with increased numbers of granulocyte-macrophage colony forming units (GM-CFUs) and blast-forming units-erythroid (BFU-Es). Such evidence of extramedullary hemopoiesis was accompanied, however, by non-significant reductions in the numbers of circulating erythrocytes and platelets. Rather, the extramedullary hemopoiesis observed by Sparwasser et al. appeared to be manifested in vivo primarily as an accelerated functional maturation of myeloid and cytotoxic T lymphocyte precursors.

In view of the foregoing, a need still exists to develop methods and compositions for treating and/or preventing anemia, thrombocytopenia, and neutropenia.

### SUMMARY OF THE INVENTION

The invention solves these and other problems by providing improved methods and compositions for treating and/or preventing anemia, thrombocytopenia, and neutropenia.

Improved methods and products for the prevention and/or treatment of anemia, thrombocytopenia, and neutropenia are provided according to the invention. The invention is based, in some aspects, on the finding that when immunostimulatory nucleic acid molecules are used in conjunction with medicaments for the treatment of anemia, thrombocytopenia, and neutropenia, some unexpected and improved results are observed. For instance, the efficacy of the combination of CpG nucleic acids with anemia, thrombocytopenia, and neutropenia medicaments is profoundly improved over the use of each of the medicaments alone. The results are surprising in part because the drugs act through different mechanisms and would not necessarily be expected to improve the efficacy of one another in a synergistic manner.

In some aspects, the invention is a method for treating or preventing anemia, thrombocytopenia, or neutropenia by administering to a subject having or at risk of developing anemia, thrombocytopenia, or neutropenia a combination of an immunostimulatory nucleic acid and an anemia, thrombocytopenia, or neutropenia medicament, wherein the combination is administered in an effective amount to treat or prevent anemia, thrombocytopenia, or neutropenia. In certain embodiments the immunostimulatory nucleic acid is a CpG nucleic acid. It was surprisingly discovered according to the invention that the combination of a CpG nucleic acid and the anemia, thrombocytopenia, or neutropenia medicament worked synergistically to promote the production of erythrocytes, platelets, or neutrophils. In certain preferred embodiments, the CpG nucleic acid is a relatively poor inducer of IFN-γ while a relatively potent inducer of other hematopoietic cytokines, e.g., IL-3, IL-6, and/or IL-12. In certain other embodiments the immunostimulatory nucleic acid is not a CpG nucleic acid, i.e., it is a non-CpG nucleic acid. In some embodiments the immunostimulatory non-CpG nucleic acid is a methylated CpG nucleic acid. In other embodiments the immunostimulatory non-CpG nucleic acid is a T-rich nucleic acid. In yet other embodiments the immunostimulatory non-CpG nucleic acid is a poly-G nucleic acid. The immunostimulatory nucleic acids of the invention can also be combinations of CpG, methylated CpG, T-rich, and/or poly-G nucleic acids. In certain embodiments the combination is encompassed in a single nucleic acid. For example, a single immunostimulatory nucleic acid might be classified simultaneously as a CpG nucleic acid and as a T-rich nucleic acid, e.g., SEQ ID NOs 70 and 77, infra. In alternative embodiments, the combination involves two or more separate nucleic acids. The methods according to this aspect of the invention are particularly useful in association with myelosuppressive treatment with irradiation or chemotherapy, because the methods reduce the risks of having or developing anemia, thrombocytopenia, or neutropenia as a side effect of the myelosuppressive treatment.

In certain embodiments the immunostimulatory nucleic acid is administered concurrently with the anemia, thrombocytopenia, or neutropenia medicament.

In certain embodiments the immunostimulatory nucleic acid has a modified backbone. The backbone in some embodiments has a phosphate modification. In certain preferred embodiments the immunostimulatory nucleic acid has a phosphorothioate backbone.

In certain embodiments the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine. In a preferred embodiment the anemia medicament is recombinant EPO.

In certain embodiments the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, and recombinant IL-11. In a preferred embodiment the thrombocytopenia medicament is recombinant TPO.

In certain embodiments the neutropenia medicament is selected from the group consisting of a glucocorticoid, recombinant G-CSF, recombinant GM-CSF, recombinant macrophage colony-stimulating factor (M-CSF), recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin. In a preferred embodiment the neutropenia medicament is recombinant G-CSF.

In this and other aspects of the invention, recombinant EPO, recombinant TPO, recombinant MGDF, recombinant G-CSF, recombinant GM-CSF, recombinant M-CSF, recombinant IL-1, recombinant IL-3, recombinant IL-6, recombinant IL-11, recombinant IFN-γ, immunoglobulin, G-CSF receptor antagonists, and IL-3 receptor antagonists may include both isolated polypeptides and isolated nucleic acids operatively linked to an expression vector which encode functional polypeptides.

In certain embodiments the subject is immunocompromised or is at risk of becoming immunocompromised. For example the subject may have received at least one dose of chemotherapy or radiation treatment. In other instances the subject may be preparing to undergo chemotherapy or radiation treatment.

In other aspects, the invention is a method for altering the dosage of the anemia, thrombocytopenia, or neutropenia medicament that is required to treat a subject suffering from anemia, thrombocytopenia, or neutropenia. The invention in one aspect is a method for increasing the dose of an anemia, thrombocytopenia, or neutropenia medicament without inducing the level of side effects ordinarily observed with that dose of an anemia, thrombocytopenia, or neutropenia medicament. The method is accomplished by administering to a subject suffering from anemia, thrombocytopenia, or neutropenia or at risk of developing anemia, thrombocytopenia, or neutropenia, an anemia, thrombocytopenia, or neutropenia medicament in a dose which would ordinarily induce side effects, administering an immunostimulatory nucleic acid to the subject, wherein administration of the immunostimulatory nucleic acid prevents the side effects associated with the high dose of the anemia, thrombocytopenia, or neutropenia medicament. The method provides a basis for administering higher therapeutic doses of an anemia, thrombocytopenia, or neutropenia medicament to a subject in order to prevent or reduce the symptoms associated with anemia, thrombocytopenia, or neutropenia more sufficiently than a lower dose. It is not desirable to administer such high doses alone, in the absence of the immunostimulatory nucleic acid, because of the side effects resulting from the high dose.

In certain embodiments of this aspect of the invention, the immunostimulatory nucleic acid is a CpG nucleic acid, while in alternative embodiments the immunostimulatory nucleic g acid is a non-CpG nucleic acid, including a methylated CpG nucleic acid, a T-rich nucleic acid, a poly-G nucleic acid, or any combination thereof as described herein.

According to this aspect of the invention, in certain embodiments the immunostimulatory nucleic acid has a modified backbone. The backbone in some embodiments has a phosphate modification. In certain preferred embodiments the immunostimulatory nucleic acid has a phosphorothioate backbone.

In certain embodiments the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine. In a preferred embodiment the anemia medicament is recombinant EPO.

In certain embodiments the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, and recombinant IL-11. In a preferred embodiment the thrombocytopenia medicament is recombinant TPO.

In certain embodiments the neutropenia medicament is selected from the group consisting of a glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin. In a preferred embodiment the neutropenia medicament is recombinant G-CSF.

In another aspect, the invention includes a method for decreasing the dose of an anemia, thrombocytopenia, or neutropenia medicament by administering to a subject having anemia, thrombocytopenia, or neutropenia or at risk of developing anemia, thrombocytopenia, or neutropenia an anemia, thrombocytopenia, or neutropenia medicament in a sub-therapeutic dosage and an immunostimulatory nucleic acid, wherein the combination of the sub-therapeutic dose of the anemia, thrombocytopenia, or neutropenia medicament and the immunostimulatory nucleic acid produce a therapeutic result in the prevention or treatment of anemia, thrombocytopenia, or neutropenia in the subject. The method permits a lower dose of the anemia, thrombocytopenia, or neutropenia medicament to be used. This provides several advantages, including lower costs associated with using lower doses of the anemia, thrombocytopenia, or neutropenia drugs and reduced chances of inducing side effects resulting from the medications by using lower doses.

In certain embodiments of this aspect of the invention, the immunostimulatory nucleic acid is a CpG nucleic acid, while in alternative embodiments the immunostimulatory nucleic acid is a non-CpG nucleic acid, including a methylated CpG nucleic acid, a T-rich nucleic acid, a poly-G nucleic acid, or any combination thereof as described herein.

According to this aspect of the invention, in certain embodiments the immunostimulatory nucleic acid has a modified backbone. The backbone in some embodiments has a phosphate modification. In certain preferred embodiments the immunostimulatory nucleic acid has a phosphorothioate backbone.

In certain embodiments the anemia medicament is selected from the group consisting of recombinant EPO, recombinant GM-CSF, recombinant G-CSF, recombinant IL-11, ferrous iron, ferric iron, vitamin B 12, vitamin B6, vitamin C, vitamin D, calcitriol, alphacalcidol, folate, androgen, and carnitine. In a preferred embodiment the anemia medicament is recombinant EPO.

In certain embodiments the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, and recombinant IL-11. In a preferred embodiment the thrombocytopenia medicament is recombinant TPO. In another preferred embodiment the thrombocytopenia medicament is a glucocorticoid. In yet another preferred embodiment the thrombocytopenia medicament comprises recombinant MGDF.

In certain embodiments the neutropenia medicament is selected from the group consisting of a glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin. In a preferred embodiment the neutropenia medicament is recombinant G-CSF.

According to yet another aspect of the invention, methods for treating or preventing anemia, thrombocytopenia, or neutropenia using specific immunostimulatory nucleic acid molecules are provided. The method in one aspect involves a method for preventing or treating anemia, thrombocytopenia, or neutropenia by administering to a subject having anemia, thrombocytopenia, or neutropenia or at risk of developing anemia, thrombocytopenia, or neutropenia, an immunostimulatory nucleic acid having a sequence selected from the group consisting of SEQ ID NO: 1 SEQ ID NO: 94 and administering to the subject an anemia, thrombocytopenia, or neutropenia medicament.

According to this aspect of the invention, in certain embodiments the immunostimulatory nucleic acid has a modified backbone. The backbone in some embodiments has a phosphate modification. In certain preferred embodiments the immunostimulatory nucleic acid has a phosphorothioate backbone.

In certain embodiments the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine.

In certain embodiments the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, and recombinant IL-11.

In certain embodiments the neutropenia medicament is selected from the group consisting of a glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin. In a preferred embodiment the neutropenia medicament is recombinant G-CSF.

According to other aspects, the invention involves methods for treating or preventing anemia, thrombocytopenia, and/or neutropenia by administering an immunostimulatory nucleic acid and an anemia, thrombocytopenia, or neutropenia medicament in different dosing schedules. In one aspect, the invention is a method for preventing or treating anemia, thrombocytopenia, or neutropenia by administering to a subject an effective amount of an immunostimulatory nucleic acid and subsequently administering to the subject an anemia, thrombocytopenia, or neutropenia medicament. In other aspects, the invention is a method for preventing or treating anemia, thrombocytopenia, or neutropenia by administering to a subject an anemia, thrombocytopenia, or neutropenia medicament and subsequently administering an immunostimulatory nucleic acid to the subject.

In certain embodiments of this aspect of the invention, the immunostimulatory nucleic acid is administered on a variable schedule, e.g., whenever the hemoglobin, hematocrit, platelet count, or neutrophil count falls to a lower cutoff level or symptoms due to anemia, thrombocytopenia, or neutropenia begin. In alternative embodiments, the immunostimulatory nucleic acid is administered on a routine schedule. A routine schedule may include every day, at least twice a week, at least three times a week, at least four times a week, at least five times a week, at least six times a week, every week, every other week, every third week, every fourth week, every month, every two months, every three months, every four months, and every six months.

In some embodiments, the immunostimulatory nucleic acid is administered consistently over a period of time, such as, for instance, in a sustained-release vehicle.

In certain embodiments of this aspect of the invention, the immunostimulatory nucleic acid is a CpG nucleic acid, while in alternative embodiments the immunostimulatory nucleic acid is a non-CpG nucleic acid, including a methylated CpG nucleic acid, a T-rich nucleic acid, a poly-G nucleic acid, or any combination thereof as described herein.

According to this aspect of the invention, in certain embodiments the immunostimulatory nucleic acid has a modified backbone. The backbone in some embodiments has a phosphate modification. In certain preferred embodiments the immunostimulatory nucleic acid has a phosphorothioate backbone.

In certain embodiments the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine.

In certain embodiments the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, and recombinant IL-11.

In certain embodiments the neutropenia medicament is selected from the group consisting of a glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin. In a preferred embodiment the neutropenia medicament is recombinant G-CSF.

The immunostimulatory nucleic acid and the anemia, thrombocytopenia, or neutropenia medicament may be administered via any effective route of administration. The immunostimulatory nucleic acid and the anemia, thrombocytopenia, or neutropenia medicament may be administered via the same or different routes. For example, either or both the immunostimulatory nucleic acid and the anemia, thrombocytopenia, or neutropenia medicament may be administered systemically, e.g., intravenously or enterally. As another example, either or both the immunostimulatory nucleic acid and the anemia, thrombocytopenia, or neutropenia medicament may be administered locally, e.g., subcutaneously, intramuscularly, mucosally, or topically.

In yet another aspect of the invention, a method for preventing or treating anemia, thrombocytopenia, or neutropenia utilizing different routes of administration is provided. In one aspect, the method involves the step of administering to a subject having anemia, thrombocytopenia, or neutropenia or at risk of developing anemia, thrombocytopenia, or neutropenia, an immunostimulatory nucleic acid, wherein the immunostimulatory nucleic acid is administered systemically and wherein the anemia, thrombocytopenia, or neutropenia medicament is administered locally. In another aspect of the invention a method is provided for treating anemia, thrombocytopenia, or neutropenia by administering to a subject having anemia, thrombocytopenia, or neutropenia or at risk of developing anemia, thrombocytopenia, or neutropenia an immunostimulatory nucleic acid and an anemia, thrombocytopenia, or neutropenia medicament, wherein the immunostimulatory nucleic acid is administered locally and the anemia, thrombocytopenia, or neutropenia medicament is administered systemically.

In certain embodiments of this aspect of the invention, the immunostimulatory nucleic acid is a CpG nucleic acid, while in alternative embodiments the immunostimulatory nucleic acid is a non-CpG nucleic acid, including a methylated CpG nucleic acid, a T-rich nucleic acid, a poly-G nucleic acid, or any combination thereof as described herein.

According to this aspect of the invention, in certain embodiments the immunostimulatory nucleic acid has a modified backbone. The backbone in some embodiments has a phosphate modification. In certain preferred embodiments the immunostimulatory nucleic acid has a phosphorothioate backbone.

In certain embodiments the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine.

In certain embodiments the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, and recombinant IL-11.

In certain embodiments the neutropenia medicament is selected from the group consisting of a glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin. In a preferred embodiment the neutropenia medicament is recombinant G-CSF.

The invention according to another aspect is a method of preventing or treating anemia, thrombocytopenia, or neutropenia by administering a poly-G nucleic acid, in an effective amount for treating or preventing anemia, thrombocytopenia, or neutropenia. In some embodiments the poly-G nucleic acid is administered alone and in other embodiments the poly-G nucleic acid is administered in conjunction with an anemia, thrombocytopenia, or neutropenia medicament. The poly-G nucleic acid in preferred embodiments comprises one of the following formulas: 5′X₁X₂GGGX₃X₄ 3′, wherein X₁, X₂, X₃, and X₄ are nucleotides, 5′ GGGNGGG 3′ or 5′ GGGNGGGNGGG 3′, wherein N represents between 0 and 20 nucleotides. In some embodiments at least one of X₃ and X₄ is a G and in other embodiments both of X₃ and X₄ are G.

The poly-G nucleic acid may be free of unmethylated CG dinucleotides, such as the nucleic acids of SEQ ID NOs 95-114, 117-121, 123-130, 132, and 133. Alternatively, the poly-G nucleic acid may include at least one unmethylated CG dinucleotide, such as the nucleic acids of SEQ ID NOs 115, 116, 122, and 131.

The invention according to another aspect is a method of preventing or treating anemia, thrombocytopenia, or neutropenia by administering a T-rich nucleic acid, in an effective amount for treating or preventing anemia, thrombocytopenia, or neutropenia. In some embodiments the T-rich nucleic acid is administered alone and in other embodiments the T-rich nucleic acid is administered in conjunction with an anemia, thrombocytopenia, or neutropenia medicament.

The invention according to another aspect is a method of preventing or treating anemia, thrombocytopenia, or neutropenia by administering a phosphorothioate backbone nucleic acid, other than a CpG nucleic acid, in an effective amount for treating or preventing anemia, thrombocytopenia, or neutropenia. In some embodiments the phosphorothioate backbone nucleic acid is administered alone and in other embodiments the T-rich nucleic acid is administered in conjunction with an anemia, thrombocytopenia, or neutropenia medicament.

A kit is provided according to another aspect of the invention. The kit in one aspect includes at least one container housing an immunostimulatory nucleic acid, at least one container housing an anemia, thrombocytopenia, or neutropenia medicament, and instructions for timing of administration of the immunostimulatory nucleic acid and the anemia, thrombocytopenia, or neutropenia medicament. In one embodiment, the the kit includes a sustained-release vehicle containing an immunostimulatory nucleic acid and at least one container housing an anemia, thrombocytopenia, or neutropenia medicament, and instructions for timing of administration of the immunostimulatory nucleic acid and the anemia, thrombocytopenia, or neutropenia medicament. In another embodiment, the kit includes containers for multiple administrations of immunostimulatory nucleic acid and at least one container housing an anemia, thrombocytopenia, or neutropenia medicament.

In certain embodiments of this aspect of the invention, the immunostimulatory nucleic acid is a CpG nucleic acid, while in alternative embodiments the immunostimulatory nucleic acid is a non-CpG nucleic acid, including a methylated CpG nucleic acid, a T-rich nucleic acid, a poly-G nucleic acid, or any combination thereof as described herein.

According to this aspect of the invention, in certain embodiments the immunostimulatory nucleic acid has a modified backbone. The backbone in some embodiments has a phosphate modification. In certain preferred embodiments the immunostimulatory nucleic acid has a phosphorothioate backbone.

In certain embodiments the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine.

In certain embodiments the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, and recombinant IL-11.

In certain embodiments the neutropenia medicament is selected from the group consisting of a glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin. In a preferred embodiment the neutropenia medicament is recombinant G-CSF.

A composition is provided according to another aspect of the invention. The composition includes an immunostimulatory nucleic acid and an anemia, thrombocytopenia, or neutropenia medicament, formulated in a pharmaceutically acceptable carrier and in an effective amount for preventing or treating anemia, thrombocytopenia, or neutropenia.

In certain embodiments of this aspect of the invention, the immunostimulatory nucleic acid is a CpG nucleic acid, while in alternative embodiments the immunostimulatory nucleic acid is a non-CpG nucleic acid, including a methylated CpG nucleic acid, a T-rich nucleic acid, a poly-G nucleic acid, or any combination thereof as described herein.

According to this aspect of the invention, in certain embodiments the immunostimulatory nucleic acid has a modified backbone. The backbone in some embodiments has a phosphate modification. In certain preferred embodiments the immunostimulatory nucleic acid has a phosphorothioate backbone.

In certain embodiments the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine.

In certain embodiments the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, and recombinant IL-11.

In certain embodiments the neutropenia medicament is selected from the group consisting of a glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin. In a preferred embodiment the neutropenia medicament is recombinant G-CSF.

Each of the limitations of the invention can encompass various embodiments of the invention. It is, therefore, anticipated that each of the limitations of the invention involving any one element or combinations of elements can be included in each aspect of the invention.

These and other aspects of the invention, as well as various advantages and utilities, will be more apparent with reference to the detailed description of the preferred embodiments and to the accompanying drawing.

All documents identified in this application are incorporated in their entirety herein by reference.

### BRIEF DESCRIPTION OF THE DRAWING

FIG. 1 depicts a kit (11) comprising an anemia, thrombocytopenia, or neutropenia medicament (17), an immunostimulatory nucleic acid in a container (19), instructions (21), and a box-like packaging (15).

### DETAILED DESCRIPTION OF THE INVENTION

The invention relates to methods and products for the treatment of anemia, thrombocytopenia, or neutropenia using a combination of immunostimulatory nucleic acids and anemia, thrombocytopenia, or neutropenia medicaments. The anemia, thrombocytopenia, or neutropenia medicaments can be administered in higher doses without as many or as severe side effects as are ordinarily encountered at those dosage levels. The anemia, thrombocytopenia, or neutropenia medicaments can also be administered in lower doses with higher efficacy than is ordinarily achieved with those doses. The immunostimulatory nucleic acids and anemia, thrombocytopenia, or neutropenia medicaments can also be administered on fixed schedules or in different temporal relationships to one another. The various combinations have many advantages over the prior art methods of treating anemia, thrombocytopenia, and neutropenia.

One method for treating or preventing anemia, thrombocytopenia, or neutropenia includes the step of administering a synergistic combination of an immunostimulatory nucleic acid and an anemia, thrombocytopenia, or neutropenia medicament in an effective amount to treat or prevent the anemia, thrombocytopenia, or neutropenia.

An “immunostimulatory nucleic acid,” as used herein, is any nucleic acid containing an immunostimulatory motif or backbone that induces an immune response. An “immune response,” as used herein, includes the stimulation of immune cells and non-immune cells to secrete or express factors which participate in and/or characterize immune activation. This term thus includes stimulation of cytokine secretion by various types of cells including lymphocytes, antigen-presenting cells, epithelial cells, and stromal cells.

Immunostimulatory motifs include, but are not limited to, CpG motifs, poly-G motifs, T-rich motifs, and combinations thereof. The CpG dinucleotides of the CpG motifs may be methylated or unmethylated. Immunostimulatory backbones include, but are not limited to, phosphate-modified backbones, such as phosphorothioate backbones. Phosphate-modified backbones can be chimeric in that they can be partly phosphodiester. Alternatively, phosphate-modified backbones can be chimeric in that they can incorporate more than one type of phosphate modification.

Certain CpG immunostimulatory nucleic acids may, because they strongly induce a Th1-like response including IFN-γ, tend to suppress hematopoiesis. Thus, as distinct from the immunostimulatory CpG nucleic acids that have been described extensively in the prior art, here the immunostimulatory nucleic acid need not promote a Th1 immune response. In fact, it is preferable that an immunostimulatory nucleic acid of the present invention does not induce the Th1 cytokine IFN-γ to a significant degree, but rather that it more characteristically promotes secretion of certain other hematopoietic cytokines, including, for example, IL-3, IL-6, and/or IL-11.

The terms “nucleic acid” and “oligonucleotide” are used interchangeably to mean multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As used herein, the terms “nucleic acid” and “oligonucleotide” refer to oligoribonucleotides as well as oligodeoxyribonucleotides. The terms shall also include polynucleosides (i.e., a polynucleotide minus the phosphate) and any other organic base-containing polymer. Nucleic acids include vectors, e.g., plasmids, as well as oligonucleotides. Nucleic acid molecules can be obtained from natural nucleic acid sources (e.g., genomic DNA or cDNA from prokaryotes including bacteria and from eukaryotes including yeast) and are referred to herein as “isolated,” but are preferably synthetic (e.g., produced by oligonucleotide synthesis).

Exemplary immunostimulatory nucleic acid sequences include but are not limited to those immunostimulatory sequences shown in Table 1. These sequences are listed without regard to methylation, i.e., they encompass both methylated and unmethylated forms.

TABLE 1

| | |
|---|---|
| GCTAGACGTTAGCGT | (SEQ ID NO: 1) |
| GCTAGATGTTAGCGT | (SEQ ID NO: 2) |
| GCTAGACGTTAGCGT | (SEQ ID NO: 3) |
| GCTAGACGTTAGCGT | (SEQ ID NO: 4) |
| GCATGACGTTGAGCT | (SEQ ID NO: 5) |
| ATGGAAGGTCCAGCGTTCTC | (SEQ ID NO: 6) |
| ATCGACTCTCGAGCGTTCTC | (SEQ ID NO: 7) |
| ATCGACTCTCGAGCGTTCTC | (SEQ ID NO: 8) |
| ATCGACTCTCGAGCGTTCTC | (SEQ ID NO: 9) |
| ATGGAAGGTCCAACGTTCTC | (SEQ ID NO: 10) |
| GAGAACGCTGGACCTTCCAT | (SEQ ID NO: 11) |
| GAGAACGCTCGACCTTCCAT | (SEQ ID NO: 12) |
| GAGAACGCTCGACCTTCGAT | (SEQ ID NO: 13) |
| GAGAACGCTGGACCTTCCAT | (SEQ ID NO: 14) |
| GAGAACGATGGACCTTCCAT | (SEQ ID NO: 15) |
| GAGAACGCTCCAGCACTGAT | (SEQ ID NO: 16) |
| TCCATGTCGGTCCTGATGCT | (SEQ ID NO: 17) |
| TCCATGTCGGTCCTGATGCT | (SEQ ID NO: 18) |
| TCCATGACGTTCCTGATGCT | (SEQ ID NO: 19) |
| TCCATGTCGGTCCTGCTGAT | (SEQ ID NO: 20) |
| TCAACGTT | (SEQ ID NO: 21) |
| TCAGCGCT | (SEQ ID NO: 22) |
| TCATCGAT | (SEQ ID NO: 23) |
| TCTTCGAA | (SEQ ID NO: 24) |
| CAACGTT | (SEQ ID NO: 25) |
| CCAACGTT | (SEQ ID NO: 26) |
| AACGTTCT | (SEQ ID NO: 27) |
| TCAACGTC | (SEQ ID NO: 28) |
| ATGGACTCTCCAGCGTTCTC | (SEQ ID NO: 29) |
| ATGGAAGGTCCAACGTTCTC | (SEQ ID NO: 30) |
| ATCGACTCTCGAGCGTTCTC | (SEQ ID NO: 31) |
| ATGGAGGCTCCATCGTTCTC | (SEQ ID NO: 32) |
| ATCGACTCTCGAGCGTTCTC | (SEQ ID NO: 33) |
| ATCGACTCTCGAGCGTTCTC | (SEQ ID NO: 34) |
| TCCATGTCGGTCCTGATGCT | (SEQ ID NO: 35) |
| TCCATGCCGGTCCTGATGCT | (SEQ ID NO: 36) |
| TCCATGGCGGTCCTGATGCT | (SEQ ID NO: 37) |
| TCCATGACGGTCCTGATGCT | (SEQ ID NO: 38) |
| TCCATGTCGATCCTGATGCT | (SEQ ID NO: 39) |
| TCCATGTCGCTCCTGATGCT | (SEQ ID NO: 40) |
| TCCATGTCGTCCCTGATGCT | (SEQ ID NO: 41) |
| TCCATGACGTGCCTGATGCT | (SEQ ID NO: 42) |
| TCCATAACGTTCCTGATGCT | (SEQ ID NO: 43) |
| TCCATGACGTCCCTGATGCT | (SEQ ID NO: 44) |
| TCCATCACGTGCCTGATGCT | (SEQ ID NO: 45) |
| GGGGTCAACGTTGACGGGG | (SEQ ID NO: 46) |
| GGGGTCAGTCGTGACGGGG | (SEQ ID NO: 47) |
| GCTAGACGTTAGTGT | (SEQ ID NO: 48) |
| TCCATGTCGTTCCTGATGCT | (SEQ ID NO: 49) |
| ACCATGGACGATCTGTTTCCCCTC | (SEQ ID NO: 50) |
| TCTCCCAGCGTGCGCCAT | (SEQ ID NO: 51) |
| ACCATGGACGAACTGTTTCCCCTC | (SEQ ID NO: 52) |
| ACCATGGACGAGCTGTTTCCCCTC | (SEQ ID NO: 53) |
| ACCATGGACGACCTGTTTCCCCTC | (SEQ ID NO: 54) |
| ACCATGGACGTACTGTTTCCCCTC | (SEQ ID NO: 55) |
| ACCATGGACGGTCTGTTTCCCCTC | (SEQ ID NO: 56) |
| ACCATGGACGTTCTGTTTCCCCTC | (SEQ ID NO: 57) |
| CACGTTGAGGGGCAT | (SEQ ID NO: 58) |
| TCAGCGTGCGCC | (SEQ ID NO: 59) |
| ATGACGTTCCTGACGTT | (SEQ ID NO: 60) |
| TCTCCCAGCGGGCGCAT | (SEQ ID NO: 61) |
| TCCATGTCGTTCCTGTCGTT | (SEQ ID NO: 62) |
| TCCATAGCGTTCCTAGCGTT | (SEQ ID NO: 63) |
| TCGTCGCTGTCTCCCCTTCTT | (SEQ ID NO: 64) |
| TCCTGACGTTCCTGACGTT | (SEQ ID NO: 65) |
| TCCTGTCGTTCCTGTCGTT | (SEQ ID NO: 66) |
| TCCATGTCGTTTTTGTCGTT | (SEQ ID NO: 67) |
| TCCTGTCGTTCCTTGTCGTT | (SEQ ID NO: 68) |
| TCCTTGTCGTTCCTGTCGTT | (SEQ ID NO: 69) |
| TCCTGTCGTTTTTTGTCGTT | (SEQ ID NO: 70) |
| TCGTCGCTGTCTGCCCTTCTT | (SEQ ID NO: 71) |
| TCGTCGCTGTTGTCGTTTCTT | (SEQ ID NO: 72) |
| TCCATGCGTGCGTGCGTTTT | (SEQ ID NO: 73) |
| TCCATGCGTTGCGTTGCGTT | (SEQ ID NO: 74) |
| TCCACGACGTTTTCGACGTT | (SEQ ID NO: 75) |
| TCGTCGTTGTCGTTGTCGTT | (SEQ ID NO: 76) |
| TCGTCGTTTTGTCGTTTTGTCGTT | (SEQ ID NO: 77) |
| TCGTCGTTGTCGTTTTGTCGTT | (SEQ ID NO: 78) |
| GCGTGCGTTGTCGTTGTCGTT | (SEQ ID NO: 79) |
| TGTCGTTTGTCGTTTGTCGTT | (SEQ ID NO: 80) |
| TGTCGTTGTCGTTGTCGTTGTCGTT | (SEQ ID NO: 81) |
| TGTCGTTGTCGTTGTCGTT | (SEQ ID NO: 82) |
| TCGTCGTCGTCGTT | (SEQ ID NO: 83) |
| TGTCGTTGTCGTT | (SEQ ID NO: 84) |
| TCCATAGCGTTCCTAGCGTT | (SEQ ID NO: 85) |
| TCCATGACGTTCCTGACGTT | (SEQ ID NO: 86) |
| GTCGYT | (SEQ ID NO: 87) |
| TGTCGYT | (SEQ ID NO: 88) |
| AGCTATGACGTTCCAAGG | (SEQ ID NO: 89) |
| TCCATGACGTTCCTGACGTT | (SEQ ID NO: 90) |
| ATCGACTCTCGAACGTTCTC | (SEQ ID NO: 91) |
| TCCATGTCGGTCCTGACGCA | (SEQ ID NO: 92) |
| TCTTCGAT | (SEQ ID NO: 93) |
| ATAGGAGGTCCAACGTTCTC | (SEQ ID NO: 94) |
| GCTAGAGGGGAGGGT | (SEQ ID NO: 95) |
| GCTAGATGTTAGGGG | (SEQ ID NO: 96) |
| GCTAGAGGGGAGGGT | (SEQ ID NO: 97) |
| GCTAGAGGGGAGGGT | (SEQ ID NO: 98) |
| GCATGAGGGGGAGCT | (SEQ ID NO: 99) |
| ATGGAAGGTCCAGGGGGCTC | (SEQ ID NO: 100) |
| ATGGACTCTGGAGGGGGCTC | (SEQ ID NO: 101) |
| ATGGACTCTGGAGGGGGCTC | (SEQ ID NO: 102) |
| ATGGACTCTGGAGGGGGCTC | (SEQ ID NO: 103) |
| ATGGAAGGTCCAAGGGGCTC | (SEQ ID NO: 104) |
| GAGAAGGGGGGACCTTCCAT | (SEQ ID NO: 105) |
| GAGAAGGGGGGACCTTCCAT | (SEQ ID NO: 106) |
| GAGAAGGGGGGACCTTGGAT | (SEQ ID NO: 107) |
| GAGAAGGGGGGACCTTCCAT | (SEQ ID NO: 108) |
| GAGAAGGGGGGACCTTCCAT | (SEQ ID NO: 109) |
| GAGAAGGGGCCAGCACTGAT | (SEQ ID NO: 110) |
| TCCATGTGGGGCCTGATGCT | (SEQ ID NO: 111) |
| TCCATGTGGGGCCTGATGCT | (SEQ ID NO: 112) |
| TCCATGAGGGGCCTGATGCT | (SEQ ID NO: 113) |
| TCCATGTGGGGCCTGCTGAT | (SEQ ID NO: 114) |
| ATGGACTCTCCGGGGTTCTC | (SEQ ID NO: 115) |
| ATGGAAGGTCCGGGGTTCTC | (SEQ ID NO: 116) |
| ATGGACTCTGGAGGGGTCTC | (SEQ ID NO: 117) |
| ATGGAGGCTCCATGGGGCTC | (SEQ ID NO: 118) |
| ATGGACTCTGGGGGGTTCTC | (SEQ ID NO: 119) |
| ATGGACTCTGGGGGGTTCTC | (SEQ ID NO: 120) |
| TCCATGTGGGTGGGGATGCT | (SEQ ID NO: 121) |
| TCCATGCGGGTGGGGATGCT | (SEQ ID NO: 122) |
| TCCATGGGGGTCCTGATGCT | (SEQ ID NO: 123) |
| TCCATGGGGGTCCTGATGCT | (SEQ ID NO: 124) |
| TCCATGTGGGGCCTGATGCT | (SEQ ID NO: 125) |
| TCCATGTGGGGCCTGATGCT | (SEQ ID NO: 126) |
| TCCATGGGGTCCCTGATGCT | (SEQ ID NO: 127) |
| TCCATGGGGTGCCTGATGCT | (SEQ ID NO: 128) |
| TCCATGGGGTTCCTGATGCT | (SEQ ID NO: 129) |
| TCCATGGGGTCCCTGATGCT | (SEQ ID NO: 130) |
| TCCATCGGGGGCCTGATGCT | (SEQ ID NO: 131) |
| GCTAGAGGGAGTGT | (SEQ ID NO: 132) |
| GGGGGGGGGGGGGGGGGGGG | (SEQ ID NO: 133) |

In some embodiments, the immunostimulatory nucleic acid is a CpG nucleic acid. CpG sequences, while relatively rare in human DNA, are commonly found in the DNA of infectious organisms such as bacteria. The human immune system has apparently evolved to recognize CpG sequences as an early warning sign of infection and to initiate an immediate and powerful immune response against invading pathogens. Thus CpG-containing nucleic acids, relying on this innate immune defense mechanism, can utilize a unique and natural pathway for immune therapy witout causing adverse reactions frequently seen with other immune stimulatory agents. The effects of CpG nucleic acids on immune modulation have been described extensively in published patent applications, such as PCT US95/01570; PCT/US97/19791; PCT/US98/03678; PCT/US98/10408; PCT/US98/04703; PCT/US99/07335; and PCT/US99/09863. The entire contents of each of these patent applications is hereby incorporated by reference.

A CpG nucleic acid is a nucleic acid which includes at least one unmethylated CpG dinucleotide. A nucleic acid containing at least one unmethylated CpG dinucleotide is a nucleic acid molecule which contains an unmethylated cytosine in a cytosine-guanine dinucleotide sequence (i.e., “CpG DNA” or DNA containing a 5′ cytosine followed by 3′ guanine and linked by a phosphate bond) and activates the immune system. The CpG nucleic acids can be double-stranded or single-stranded. Generally, double-stranded molecules are more stable in vivo, while single-stranded molecules have increased immune activity. Thus in some aspects of the invention it is preferred that the nucleic acid be single-stranded and in other aspects it is preferred that the nucleic acid be double-stranded. The terms CpG nucleic acid or CpG oligonucleotide as used herein refer to an immunostimulatory CpG nucleic acid or immunostimulatory CpG oligonucleotide unless otherwise indicated. The entire immunostimulatory CpG nucleic acid can be unmethylated or portions may be unmethylated, but at least the C of the 5′-CG-3′ dinucleotide must be unmethylated.

In a preferred embodiment, a CpG nucleic acid is further characterized by its relatively poor ability, compared to other CpG nucleic acids selected specifically for their ability to induce the secretion of IFN-γ and to favor a Th1-like immune response, to induce IFN-γ relative to hematopoietic cytokines such as IL-3, IL-6, IL-11, IL-12, G-CSF, and/or GM-CSF. Determination of the relative ability to induce these hematopoietic cytokines can be readily determined using conventional in vitro assay methods such as enzyme-linked immunosorbent assay (ELISA) and/or fluorescence-activated cell sorting (FACS) analysis. The latter method is particularly useful for performing assays of cytokines located intracellularly, e.g., IL-12 and IFN-γ.

The context of the CpG dinucleotide, i.e., the CpG motif, may in some instances be at least as important as the methylation. Thus in another embodiment, an immunostimulatory nucleic acid may have all the characteristics of a CpG nucleic acid as described above, with the exception that the C of the at least one CpG dinucleotide need not be unmethylated. As used herein, a “methylated CpG nucleic acid” refers to an immunostimulatory nucleic acid having all the characteristics of a CpG nucleic acid as described above, with the exception that the C of the at least one CpG dinucleotide is methylated. Such an immunostimulatory nucleic acid may include at least one methylated CpG dinucleotide and at least one unmethylated CpG dinucleotide. In an alternative embodiment, the entire immunostimulatory nucleic acid can be methylated, including all CpG dinucleotides present.

In one preferred embodiment the invention provides an immunostimulatory nucleic acid which is a CpG nucleic acid represented by at least the formula:

5′X₁X₂CGX₃X₄ 3′

wherein X₁, X₂, X₃, and X₄ are nucleotides. In one embodiment X₂ is adenine, guanine, cytosine, or thymine. In another embodiment X₃ is adenine, guanine, cytosine, or thymine. In other embodiments X₂ is guanine, adenine, or thymine and X₃ is cytosine, adenine, or thymine.

In another embodiment the immunostimulatory nucleic acid is an isolated CpG nucleic acid represented by at least the formula:

5′₁X₁X₂CGX₃X₄N₂ 3′

wherein X₁, X₂, X₃, and X₄ are nucleotides, N is any nucleotide, and N₁ and N₂ are nucleic acid sequences composed of from about 0-25 N's each. In one embodiment X₁X₂ is a dinucleotide selected from the group consisting of: GpG, GpA, GpT, ApG, ApA, ApT, CpG, CpA, CpT, TpG, TpA, and TpT; and X₃X₄ is a dinucleotide selected from the group consisting of: ApG, ApA, ApT, ApC, CpG, CpA, CpC, TpG, TpA, TpT, and TpC. Preferably X₁X₂ is GpA or GpT and X₃X₄ is TpT. In other embodiments X₁ or X₂ or both are purines and X₃ or X₄ or both are pyrimidines or X₁X₂ is GpA and X₃ or X₄ or both are pyrimidines. In another preferred embodiment X₁X₂ is a dinucleotide selected from the group consisting of: TpA, ApA, ApC, ApG, and GpG. In yet another embodiment X₃X₄ is a dinucleotide selected from the group consisting of: ApG, ApA, ApC, TpG, TpA, TpT, and CpA. X₁X₂ in another embodiment is a dinucleotide selected from the group consisting of: GpT, GpC, ApT, TpG, TpT, TpC, CpG, CpT, and CpC.

In another preferred embodiment the immunostimulatory nucleic acid has the sequence 5′TCN₁TX₁X₂CGX₃X₄ 3′. The immunostimulatory nucleic acids of the invention in some embodiments include X₁X₂ selected from the group consisting of GpG, GpA, GpT, and ApA and X₃X₄ is selected from the group consisting of TpT, TpC, and CpT.

For facilitating uptake into cells, the immunostimulatory nucleic acids are preferably in the range of 6 to 100 bases in length. However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

“Palindromic sequence” shall mean an inverted repeat (i.e., a sequence such as ABCDEE′D′C′B′A′ in which A and A′ are bases capable of forming the usual Watson-Crick base pairs). In vivo, such sequences may form double-stranded structures. In one embodiment the CpG nucleic acid contains a palindromic sequence. A palindromic sequence used in this context refers to a palindrome in which the CpG is part of the palindrome, and preferably is the center of the palindrome. In another embodiment the CpG nucleic acid is free of a palindrome. An immunostimulatory nucleic acid that is free of a palindrome is one in which the CpG dinucleotide is not part of a palindrome. Such an oligonucleotide may include a palindrome in which the CpG is not the center of the palindrome.

The CpG nucleic acid sequences of the invention are those broadly described above as well as disclosed in PCT Published Patent Applications PCT/US95/01570 and PCT/US97/19791 claiming priority to U.S. Ser. Nos. 08/386,063 and 08/960,774, filed on Feb. 7, 1995 and Oct. 30, 1997, respectively.

The immunostimulatory nucleic acids of the invention also include nucleic acids having T-rich motifs. As used herein, “T-rich nucleic acid” refers to a nucleic acid which includes at least one poly-T sequence and/or which has a nucleotide composition of greater than 25 percent thymine (T) nucleotide residues. A poly-T sequence includes at least four consecutive T nucleotides and does not require the presence of a CpG motif. A T-rich nucleic acid may optionally be free of unmethylated CpG dinucleotides or free of methylated CpG dinucleotides. It was recently discovered by Dr. Arthur Krieg that T-rich nucleic acids are immunostimulatory. It was presented by Dr. Krieg at the International Workshop on “Immunobiology of Bacterial CpG-DNA” held in Upper Bavaria on Sep. 26-29, 1999, that poly-T nucleic acids of 24 bases in length are immunostimulatory, whereas the same length poly-C oligonucleotide is non-immunostimulatory. These concepts are also described and claimed in U.S. Provisional Patent Application No. 60/156,113 filed on Sep. 25, 1999, and U.S. patent application Ser. No. 09/669,187, filed on Sep. 25, 2000, the entire contents of which are hereby incorporated by reference.

A number of references also describe the immunostimulatory properties of poly-G nucleic acids (defined below). Pisetsky and Reich (1993) Mol Biol Reports 18:217-221; Krieger and Herz (1994) Ann Rev Biochem 63:601-637; Macaya et al. (1993) Proc Natl Acad Sci USA 90:3745-3749; Wyatt et al. (1994) Proc Natl Acad Sci USA 91:1356-1360; Rando and Hogan (1998) In: Applied Antisense Oligonucleotide Technology, eds. Krieg and Stein, p. 335-352; and Kimura et al. (1994) J Biochem 116:991-994. Poly-G-containing oligonucleotides are useful for treating and preventing bacterial and viral infections.

In some aspects of the invention the poly-G containing nucleic acids are administered alone for the treatment of anemia, thrombocytopenia, and neutropenia. It was previously suggested in the prior art that poly-G rich oligonucleotides inhibit the production of IFN-γ by compounds such as CpG oligonucleotides, concanavalin A, bacterial DNA, or the combination of phorbol 12-myristate 13-acetate (PMA) and the calcium ionophore A 23187 (Halperin and Pisetsky (1995) Immunopharmacol 29:47-52), as well as block the downstream effects of IFN-γ. For instance, Ramanathan et al. has shown that a poly-G oligonucleotide inhibits the binding of IFN-γ to its receptor, which prevents the normal enhancement of MHC class I and ICAM-1 in response to IFN-γ. Ramanathan et al. (1994) Transplantation 57:612-615. Poly-G oligonucleotides were also found to be able to inhibit the secretion of IFN-γ from lymphocytes. Halperin and Pisetsky (1995) Immunopharmacol 29:47-52. It was surprisingly discovered according to the invention that when poly-G nucleic acids are administered in vivo, they are useful for treating or preventing anemia, thrombocytopenia, or neutropenia. Thus, in this aspect of the invention, poly-G nucleic acids are administered alone or optionally with other anemia, thrombocytopenia, or neutropenia medicaments for the treatment of anemia, thrombocytopenia, and/or neutropenia.

Poly-G nucleic acids preferably are nucleic acids having the following formula:

5′X₁X₂GGGX₃X₄ 3′

wherein X₁, X₂, X₃, and X₄ are nucleotides. In preferred embodiments at least one of X₃ and X₄ is a G. In other embodiments both of X₃ and X₄ are G's. In yet other embodiments the preferred formula is 5′ GGGNGGG 3′ or 5GGGNGGGNGGG 3′, wherein N represents between 0 and 20 nucleotides. In other embodiments the poly-G nucleic acid is free of unmethylated CG dinucleotides, such as, for example, the nucleic acids listed above as SEQ ID NOs 95-114, 117-121, 123-130, 132, and 133. In other embodiments the poly-G nucleic acid includes at least one unmethylated CG dinucleotide, such as, for example, the nucleic acids listed above as SEQ ID NOs 115, 116, 122, and 131.

Nucleic acids having modified backbones, such as phosphorothioate backbones, fall within the class of immunostimulatory nucleic acids. U.S. Pat. Nos. 5,723,335 and 5,663,153 issued to Hutcherson et al. and related PCT publication WO95/26204 describe immune stimulation using phosphorothioate oligonucleotide analogues. These patents describe the ability of the phosphorothioate backbone to stimulate an immune response in a non-sequence-specific manner.

In the case when the immunostimulatory nucleic acid is administered in conjunction with a nucleic acid vector, under certain circumstances it is useful if the backbone of the immunostimulatory nucleic acid is a chimeric combination of phosphodiester and phosphorothioate (or other phosphate modification). The cell may have a problem taking up a plasmid vector in the presence of completely phosphorothioate oligonucleotide. Thus when both a vector and an oligonucleotide are delivered to a subject, it is preferred that the oligonucleotide have a chimeric backbone, or, if the oligonucleotide has a completely phosphorothioate backbone, that the plasmid is associated with a vehicle that delivers it directly into the cell, thus avoiding the need for cellular uptake. Such vehicles are known in the art and include, for example, liposomes and gene guns.

In the case when more than one immunostimulatory nucleic acid is administered, either alone or in conjunction with a vector, the backbone of one immunostimulatory nucleic acid can be completely phosphorothioate and the backbone of another immunostimulatory nucleic acid completely phosphodiester. Thus, for example, a phosphorothioate ODN may be given together with a phosphodiester ODN.

For use in the instant invention, the immunostimulatory nucleic acids can be synthesized de novo using any of a number of procedures well known in the art. Such compounds are referred to as “synthetic nucleic acids.” These methods of synthesis include, for example, the β-cyanoethyl phosphoramidite method (Beaucage S L and Caruthers M H is (1981) Tetrahedron Lett 22:1859), and the nucleoside H-phosphonate method (Garegg et al. (1986) Tetrahedron Lett 27:4051-4054; Froehler et al. (1986) Nucl Acid Res 14:5399-5407; Garegg et al. (1986) Tetrahedron Lett 27:4055-4058; Gaffney et al. (1988) Tetrahedron Lett 29:2619-2622). These chemistries can be performed by a variety of automated oligonucleotide synthesizers available in the market. These nucleic acids are referred to as synthetic nucleic acids. Alternatively, immunostimulatory nucleic acids can be produced on a large scale in plasmids (see Sambrook et al., “Molecular Cloning: A Laboratory Manual,” Cold Spring Harbor Laboratory Press, New York (1989)) and separated into smaller pieces or administered whole. Nucleic acids can be prepared from natural nucleic acid sequences (e.g., genomic DNA or cDNA) using known techniques, such as those employing restriction enzymes, exonucleases or endonucleases. Nucleic acids prepared in this manner are referred to as isolated nucleic acids. The term “immunostimulatory nucleic acid” encompasses both synthetic and isolated immunostimulatory nucleic acids.

For use in vivo, nucleic acids are preferably relatively resistant to degradation (e.g., are stabilized). A “stabilized nucleic acid molecule” shall mean a nucleic acid molecule that is relatively resistant to in vivo degradation (e.g., via an exo- or endonuclease). Stabilization can be a function of length or secondary structure. Immunostimulatory nucleic acids that are tens to hundreds of kbs long are relatively resistant to in vivo degradation. For shorter immunostimulatory nucleic acids, secondary structure can stabilize and increase their effect. For example, if the 3′ end of a nucleic acid has self-complementarity to an upstream region, so that it can fold back and form a sort of stem-loop structure, then the nucleic acid becomes stabilized and therefore exhibits more activity.

Alternatively, nucleic acid stabilization can be accomplished via backbone modifications. Preferred stabilized nucleic acids of the instant invention have a modified backbone. It has been demonstrated that modification of the nucleic acid backbone provides enhanced activity of the immunostimulatory nucleic acids when administered in vivo. One type of modified backbone is a phosphate backbone modification. Inclusion in immunostimulatory nucleic acids of at least two phosphorothioate linkages at the 5′ end of the oligonucleotide and multiple (preferably five) phosphorothioate linkages at the 3′ end, can in some circumstances provide maximal activity and protect the nucleic acid from degradation by intracellular exo- and endonucleases. Other phosphate-modified nucleic acids include phosphodiester-modified nucleic acids, combinations of phosphodiester and phosphorothioate nucleic acids, methylphosphonate, methylphosphorothioate, phosphorodithioate, and combinations thereof. Each of these combinations in CpG nucleic acids and their particular effects on immune cells is discussed in more detail in PCT Published Patent Applications PCT/US95/01570 and PCT/US97/19791, the entire contents of which are hereby incorporated by reference. Although Applicants are not bound by the theory, it is believed that these phosphate-modified nucleic acids may show more stimulatory activity due to enhanced nuclease resistance, increased cellular uptake, increased protein binding, and/or altered intracellular localization.

Modified backbones such as phosphorothioates may be synthesized using automated techniques employing either phosphoramidate or H-phosphonate chemistries as described above. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described. Uhlmann E and Peyman A (1990) Chem Rev 90:544; Goodchild J (1990) Bioconjugate Chem 1:165.

Both phosphorothioate and phosphodiester nucleic acids containing immunostimulatory motifs are active in immune cells. However, based on the concentration needed to induce immunostimulatory nucleic acid specific effects, the nuclease-resistant phosphorothioate backbone immunostimulatory nucleic acids are more potent. In certain in vitro assays, phosphorothioate CpG is two orders of magnitude more potent than phosphodiester CpG. Krieg A M et al. (1995) Nature 374:546-549.

Another type of modified backbone, useful according to the invention, is a peptide nucleic acid (PNA). The backbone is composed of aminoethylglycine and supports bases which provide the DNA-like character. The backbone does not include any phosphate and thus may optionally have no net charge. The lack of charge allows for stronger DNA-DNA binding because the charge repulsion between the two strands does not exist. Additionally, because the backbone has an extra methylene group, the oligonucleotides are enzyme/protease resistant. PNAs can be purchased from various commercial sources, e.g., Perkin Elmer, or synthesized de novo.

Another class of backbone modifications include 2′-O-methylribonucleosides (2′-Ome). These types of substitutions are described extensively in the prior art and in particular with respect to their immunostimulating properties in Zhao Q et al. (1999) Bioorg Med Chem Lett 9:3453-8. Zhao et al. describes methods of preparing 2′-Ome modifications to nucleic acids.

The nucleic acid molecules of the invention may include naturally occurring or synthetic purine or pyrimidine heterocyclic bases as well as modified backbones. Purine or pyrimidine heterocyclic bases include, but are not limited to, adenine, guanine, cytosine, thymine, uracil, and inosine. Other representative heterocyclic bases are disclosed in U.S. Pat. No. 3,687,808, issued to Merigan, et al. The term purine or pyrimidine or bases are used herein to refer to both naturally occurring and synthetic purines, pyrimidines or bases.

Other stabilized nucleic acids include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The immunostimulatory nucleic acids having backbone modifications useful according to the invention in some embodiments are S- or R-chiral immunostimulatory nucleic acids. An “S-chiral immunostimulatory nucleic acid” as used herein is an immunostimulatory nucleic acid wherein at least two nucleotides have a backbone modification forming a chiral center and wherein a plurality of the chiral centers have S chirality. An “R-chiral immunostimulatory nucleic acid” as used herein is an immunostimulatory nucleic acid wherein at least two nucleotides have a backbone modification forming a chiral center and wherein a plurality of the chiral centers have R chirality. The backbone modification may be any type of modification that forms a chiral center. The modifications include but are not limited to phosphorothioate, methylphosphonate, methylphosphorothioate, phosphorodithioate, 2′-Ome and combinations thereof.

The chiral immunostimulatory nucleic acids must have at least two nucleotides within the nucleic acid that have a backbone modification. All or less than all of the nucleotides in the nucleic acid, however, may have a modified backbone. Of the nucleotides having a modified backbone (referred to as chiral centers), a plurality have a single chirality, S or R. A “plurality” as used herein refers to an amount greater than or equal to 75%. Thus, less than all of the chiral centers may have S or R chirality as long as a plurality of the chiral centers have S or R chirality. In some embodiments at least 75%, 80%, 85%, 90%, 95%, or 100% of the chiral centers have S or R chirality. In other embodiments at least 75%, 80%, 85%, 90%, 95%, or 100% of the nucleotides have backbone modifications.

The S- and R-chiral immunostimulatory nucleic acids may be prepared by any method known in the art for producing chirally pure oligonucleotides. Stec et al. teaches methods for producing stereopure phosphorothioate oligodeoxynucleotides using an oxathiaphospholane. Stec W J et al. (1995) J Am Chem Soc 117:12019. Other methods for making chirally pure oligonucleotides have been described by companies such as ISIS Pharmaceuticals. U.S. Patents have also described these methods. For instance U.S. Pat. Nos. 5,883,237; 5,856,465; 5,837,856; 5,599,797; 5,521,302; 5,512,668; 5,506,212; 5,359,052; and 5,212,295, each of which is hereby incorporated by reference in its entirety, disclose methods for generating stereopure oligonucleotides.

The immunostimulatory nucleic acids are useful for treating or preventing anemia, thrombocytopenia, or neutropenia in a subject. A “subject” shall mean a human or vertebrate mammal including but not limited to a dog, cat, horse, cow, pig, sheep, goat, or primate, e.g., monkey.

The immunostimulatory nucleic acids are useful in some aspects of the invention as a prophylactic for the treatment of a subject at risk of developing anemia, thrombocytopenia, or neutropenia. For example, the immunostimulatory nucleic acids can be administered to a subject where it is anticipated that the subject will be exposed to conditions associated with development of anemia, thrombocytopenia, or neutropenia. Alternatively, the immunostimulatory nucleic acids can be administered to a subject where it is known or suspected that the subject is predisposed to develop anemia, thrombocytopenia, or neutropenia. A “subject at risk” of developing anemia, thrombocytopenia, or neutropenia as used herein is a subject who has any risk of exposure to an agent associated with suppression of formation of erythrocytes, platelets, neutrophils, or their progenitors, risk of loss of erythrocytes, platelets, or neutrophils associated with surgery or injury, or risk of developing anemia, thrombocytopenia, or neutropenia by some other mechanism involving diminished production, accelerated destruction, and/or sequestration of erythrocytes, platelets, or neutrophils, e.g., autoimmune destruction of erythrocytes or platelets, thalassemia, and renal insufficiency.

In addition to the use of the immunostimulatory nucleic acid and the anemia, thrombocytopenia, or neutropenia medicament for prophylactic treatment, the invention also encompasses the use of the combination of drugs for the treatment of a subject having anemia, thrombocytopenia, or neutropenia. A “subject having anemia” is a subject that has a reduced number of circulating erythrocytes. A “subject having thrombocytopenia” is a subject that has a reduced number of circulating platelets. A “subject having neutropenia” is a subject that has a reduced number of circulating neutrophils (also known as granulocytes, polymorphonuclear leukocytes, PMNs).

Anemia, thrombocytopenia, and neutropenia are frequently defined in terms of laboratory measurements indicating a reduced hematocrit (volume percent), a reduced platelet count (per mm³), and a reduced neutrophil count (per mm³), respectively. Methods of determining these values are well known in the art, including automated as well as manual methods. The lower limits of normal for hematocrits and platelet counts in healthy nonpregnant humans is somewhat variable, depending on the age and sex of the subject, method of determination, and the norms for the laboratory performing the mesurements. Generally, however, an adult human subject is said to have anemia when the hematocrit is less than about 37-40%. Likewise, generally an adult human subject is said to have thrombocytopenia when the platelet count is below about 100,000 per mm³. Anemia is also frequently reported in terms of a reduced hemoglobin (g/dL) or red blood cell count (per mm³). Typical lower limits of normal values for these in healthy adult humans are 12-13 g/dL and about 4.1×10⁶ per mm³ , respectively. Generally an adult human subject is said to have neutropenia when the neutrophil count falls below 1000 per mm³. Corresponding values for all these parameters are different for other species.

Anemia, thrombocytopenia, and neutropenia are also frequently associated with clinical signs and symptoms in relation to their degree of severity. Anemia may be manifested as pallor, generalized fatigue or weakness, reduced exercise tolerance, shortness of breath with exertion, rapid heart rate, irregular heart rhythm, chest pain (angina), congestive heart failure, and headache. Thrombocytopenia is typically manifested in terms of spontaneous or uncontrolled bleeding, petechiae, and easy bruising. Neutropenia is associated with infections, including notably infections from endogenous microbial flora, and lack of inflammation.

An “anemia medicament” as used herein is a composition of matter which reduces the symptoms related to anemia, prevents the development of anemia, or treats existing anemia.

A “thrombocytopenia medicament” as used herein is a composition of matter which reduces the symptoms related to thrombocytopenia, prevents the development of thrombocytopenia, or treats existing thrombocytopenia.

A “neutropenia medicament” as used herein is a composition of matter which reduces the symptoms related to neutropenia, prevents the development of neutropenia, or treats existing neutropenia.

The anemia, thrombocytopenia, or neutropenia medicaments useful in combination with the immunostimulatory nucleic acids include steroids, inducers of steroids, and immunomodulators.

The steroids include, but are not limited to, systemically administered corticosteroids including methylprednisolone, prednisolone and prednisone, cortisone, and hydrocortisone. Inducers of steroids include, but are not limited to adrenocorticotropic hormone (ACTH).

Corticosteroids inhibit cytokine production, adhesion protein activation, and inflammatory cell migration and activation. The side effects associated with systemic corticosteroids include, for instance, reversible abnormalities in glucose metabolism, increased appetite, fluid retention, weight gain, mood alteration, hypertension, peptic ulcer, and asceptic necrosis of bone. Some side effects associated with longer term use include adrenal axis suppression, growth suppression, dermal thinning, hypertension, diabetes mellitus, Cushing's syndrome, cataracts, muscle weakness, and in rare instances, impaired immune function. It is recommended that these types of compounds be used at their lowest effective dose.

Commonly used anemia drugs which are currently on the market or in development include recombinant human EPO (EPOGEN; PROCRIT), preparations of iron (ferrous and ferric, CHROMAGEN; FEOSOL; INFED; IROSPAN; NEPHRO-FER; NEPHRO-VITE; NIFEREX; NU-IRON; SLOW FE), vitamin B12, vitamin B6, folic acid (CHROMAGEN; FERRO-FOLIC; NEPHRO-FER; NIFEREX), ascorbic acid, certain metabolites of vitamin D (calcitriol and alphacalcidol; CALCIJEX; ROCALTROL), androgens, anabolic steroids (ANADROL), camitine, recombinant IL-11 (NEUMEGA), and G-CSF (NEUPOGEN). In a preferred embodiment the anenia medicament is recombinant EPO.

Drugs in common usage or development for the treatment of thrombocytopenia include glucocorticoids (prednisolone; prednisone; methylprednisolone; SOLUMEDROL), recombinant TPO, recombinant MGDF, pegylated recombinant MGDF, lisophylline, recombinant IL-1, recombinant IL-3, recombinant IL-6, recombinant IL-11 (NEUMEGA), and recombinant G-CSF (NEUPOGEN). In a preferred embodiment the thrombocytopenia medicament is recombinant TPO.

Drugs in common usage or development for the treatment of neutropenia include glucocorticoids (prednisolone; prednisone; methylprednisolone; SOLUMEDROL), recombinant G-CSF (NEUPOGEN), recombinant GM-CSF (LEUKINE), recombinant M-CSF, recombinant IL-1, recombinant IL-3, recombinant IL-6, immunoglobulin G (SANDOGLOBULIN, IVEEGAM, GAMMAR-P, GAMIMNE N, GAMMAGARD S/D), androgens, recombinant IFN-γ (ACTIMMUNE), small molecule G-CSF mimetics, G-CSF receptor antagonists, IL-3 receptor antagonists, and uteroferrin. In a preferred embodiment the neutropenia medicament is recombinant G-CSF. Antibiotics are frequently adminstered in association with neutropenia medicaments to treat or reduce the risk of infection.

As used herein, the term “prevent”, “prevented”, or “preventing”, when used with respect to the treatment of anemia, thrombocytopenia, or neutropenia, refers to a prophylactic treatment which increases the resistance of a subject to developing anemia, thrombocytopenia, or neutropenia or, in other words, decreases the likelihood that the subject will develop anemia, thrombocytopenia, or neutropenia as well as a treatment after the anemia, thrombocytopenia, or neutropenia has begun in order to reduce or eliminate it altogether or prevent it from becoming worse.

The term “substantially purified” as used herein refers to a molecular species which is substantially free of other proteins, lipids, carbohydrates or other materials with which it is naturally associated.

The immunostimulatory nucleic acids may also be delivered to the subject in the form of a plasmid vector. In some embodiments, one plasmid vector could include both the immunostimulatory nucleic acid and a nucleic acid encoding a polypeptide anemia, thrombocytopenia, or neutropenia medicament. In other embodiments, separate plasmids could be used. In yet other embodiments, no plasmids could be used.

The compositions of the invention may be delivered to the immune system or other target cells alone or in association with a vector. In its broadest sense, a “vector” is any vehicle capable of facilitating the transfer of the compositions to the target cells. The vector generally transports the nucleic acid to the immune cells with reduced degradation relative to the extent of degradation that would result in the absence of the vector.

In general, the vectors useful in the invention are divided into two classes: biological vectors and chemical/physical vectors. Biological vectors and chemical/physical vectors are useful for delivery and/or uptake of nucleic acids, anemia, thrombocytopenia, or neutropenia medicaments to/by a target cell.

Biological vectors include, but are not limited to, plasmids, phagemids, viruses, other vehicles derived from viral or bacterial sources that have been manipulated by the insertion or incorporation of nucleic acid sequences, and free nucleic acid fragments which can be attached to nucleic acid sequences. Viral vectors are a preferred type of biological vector and include, but are not limited to, nucleic acid sequences from the following viruses: retroviruses, such as: Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One can readily employ other viral vectors not named but known in the art.

Preferred viral vectors are based on non-cytopathic eukaryotic viruses in which non-essential genes have been replaced with a nucleic acid of interest. Non-cytopathic viruses include retroviruses, the life cycle of which involves reverse transcription of genomic viral RNA into DNA with subsequent proviral integration into host cellular DNA. Retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Cliffton, N.J. (1991).

Another preferred virus for certain applications is the adeno-associated virus (AAV), a double-stranded DNA virus. The AAV can be engineered to be replication-deficient and is capable of infecting a wide range of cell types and species. It further has advantages, such as heat and lipid solvent stability; high transduction frequencies in cells of diverse lineages; and lack of superinfection inhibition, thus allowing multiple series of transductions. Reportedly, the AAV can integrate into human cellular DNA in a site-specific manner, thereby minimizing the possibility of insertional mutagenesis and variability of inserted gene expression. In addition, wild-type AAV infections have been followed in tissue culture for greater than 100 passages in the absence of selective pressure, implying that the AAV genomic integration is a relatively stable event. The AAV can also function in an extrachromosomal fashion.

Other biological vectors include plasmid vectors. Plasmid vectors have been extensively described in the art and are well known to those of skill in the art. See e.g., Sambrook et al., “Molecular Cloning: A Laboratory Manual,” Second Edition, Cold Spring Harbor Laboratory Press (1989). In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operatively encoded within the plasmid. Some commonly used plasmids include pBR322, pUC18, pUC19, pRc/CMV, pCDNA3.1, pSV40, and pBlueScript. Other plasmids are well-known to those of ordinary skill in the art. Additionally, plasmids may be custom designed using restriction enzymes and ligation reactions to remove and add specific fragments of DNA.

It has recently been discovered that gene-carrying plasmids can be delivered to the immune system using bacteria. Modified forms of bacteria such as Salmonella can be transfected with the plasmid and used as delivery vehicles. The bacterial delivery vehicles can be administered to a host subject orally or by other administration means. The bacteria deliver the plasmid to immune cells, e.g., B cells and dendritic cells, likely by passing through the gut barrier. High levels of immune protection have been established using this methodology. Such methods of delivery are useful for the aspects of the invention utilizing systemic delivery of immunostimulatory nucleic acid and/or other therapeutic agent.

In addition to the biological vectors, chemical/physical vectors may be used to deliver a nucleic acid, anemia medicament, thrombocytopenia medicament, and/or neutropenia medicament to a target cell and facilitate uptake thereby. As used herein, a “chemical/physical vector” refers to a natural or synthetic molecule, other than those derived from bacteriological or viral sources, capable of delivering the nucleic acid, anemia, thrombocytopenia, or neutropenia medicament, and/or other therapeutic agent to a cell.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vesicles (LUV), which range in size from 0.2-4.0 μm can encapsulate large macromolecules. RNA, DNA, and intact virions can be encapsulated within the aqueous interior and be delivered to cells in a biologically active form. Fraley et al. (1981) Trends Biochem Sci 6:77.

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2, 3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnology 3:235-241.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”). PCT/US/03307 describes a biocompatible, preferably if biodegradable polymeric matrix for containing an exogenous gene under the control of an appropriate promoter. The polymeric matrix can be used to achieve sustained release of the exogenous gene in the patient.

The polymeric matrix preferably is in the form of a microparticle such as a microsphere (wherein the nucleic acid, anemia, thrombocytopenia, or neutropenia medicament, and/or other therapeutic agent is dispersed throughout a solid polymeric matrix) or a microcapsule (wherein the a nucleic acid, anemia, thrombocytopenia, or neutropenia medicament, and/or other therapeutic agent is stored in the core of a polymeric shell). Other forms of the polymeric matrix for containing the a nucleic acid, anemia, thrombocytopenia, or neutropenia medicament, and/or other therapeutic agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, for instance injection into a tissue or administration of a suspension by aerosol into the nasal and/or pulmonary areas. Preferably when an aerosol route is used the polymeric matrix and the nucleic acid, anemia, thrombocytopenia, or neutropenia medicament, and/or other therapeutic agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time.

In another embodiment the chemical/physical vector is a biocompatible microsphere that is suitable for delivery, such as oral or mucosal delivery. Such microspheres are disclosed in Chickering et al. (1996) Biotech Bioeng 52:96-101 and Mathiowitz et al. (1997) Nature 386:410-414 and PCT Patent Application WO97/03702.

Both non-biodegradable and biodegradable polymeric matrices can be used to deliver the nucleic acid, anemia, thrombocytopenia, or neutropenia medicament, and/or other therapeutic agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is crosslinked with multivalent ions or other polymers.

Bioadhesive polymers of particular interest include bioerodible hydrogels described by H. S. Sawhney, C. P. Pathak and J. A. Hubell in Macromolecules (1993) 26:581-587, the teachings of which are incorporated herein. Such bioerodible hydrogels include those formed on the basis of, for example, polyhyaluronic acids, casein, gelatin, gluten, polyanhydrides, polyacrylic acid, alginate, chitosan, poly(methyl methacrylates), poly(ethyl methacrylates), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake by a target cell of the nucleic acid include calcium phosphate and other chemical mediators of intracellular transport, microinjection compositions, and electroporation.

The immunostimulatory nucleic acid and/or the anemia, thrombocytopenia, or neutropenia medicament may be administered alone (e.g., in saline or buffer) or using any delivery vectors known in the art. For instance the following delivery vehicles have been described: cochleates (Gould-Fogerite et al. (1994, 1996)); emulsomes (Vancott et al. (1998), Lowell et al. (1997)); ISCOMs (Mowat et al. (1993), Carlsson et al. (1991), Hu et. (1998), Morein et al. (1999)); liposomes (Childers et al. (1999), Michalek et al. (1989, 1992), de Haan (1995a, 1995b)); live bacterial vectors (e.g., Salmonella, Escherichia coli, Bacillus Calmette-Guérin, Shigella, Lactobacillus) (Hone et al. (1996), Pouwels et al. (1998), Chatfield et al. (1993), Stover et al. (1991), Nugent et al. (1998)); live viral vectors (e.g., vaccinia, adenovirus, herpes simplex) (Gallichan et al. (1993, 1995), Moss et al. (1996), Nugent et al. (1998), Flexner et al. (1988), Morrow et al. (1999)); microspheres (Gupta et al. (1998), Jones et al. (1996), Maloy et al. (1994), Moore et al. (1995), O'Hagan et al. (1994), Eldridge et al. (1989)); nucleic acid vaccines (Fynan et al. (1993), Kuklin et al. (1997), Sasaki et al. (1998), Okada et al. (1997), Ishii et al. (1997)); polymers (e.g., carboxymethylcellulose, chitosan) (Hamajima et al. (1998), Jabbal-Gill et al. (1998)); polymer rings (Wyatt et al. (1998)); proteosomes (Vancott et al. (1998), Lowell et al. (1988, 1996, 1997)); sodium fluoride (Hashi et al. (1998)); transgenic plants (Tacket et al. (1998), Mason et al. (1998), Haq et al. (1995)); virosomes (Gluck et al. (1992), Mengiardi et al. (1995), Cryz et al. (1998)); virus-like particles (Jiang et al. (1999), Leibl et al. (1998)).

The immunostimulatory nucleic acid and anemia, thrombocytopenia, or neutropenia medicament can be combined with other therapeutic agents such as adjuvants to enhance hematopoiesis even further. Other therapeutic agents here include but are not limited to non-nucleic acid adjuvants, cytokines (i.e., other than the anemia, thrombocytopenia, or neutropenia medicament), antibodies, antigens, etc. The immunostimulatory nucleic acid, anemia, thrombocytopenia, or neutropenia medicament and other therapeutic agent may be administered simultaneously or sequentially. When the other therapeutic agents are administered simultaneously, they can be administered in the same or separate formulations, but are administered at the same time. The other therapeutic agents are administered sequentially with one another and with the immunostimulatory nucleic acid and anemia, thrombocytopenia, or neutropenia medicament, when the administration of the other therapeutic agents and the immunostimulatory nucleic acid and anemia, thrombocytopenia, or neutropenia medicament is temporally separated. The separation in time between the administration of these compounds may be a matter of seconds or it may be longer.

A “non-nucleic acid adjuvant” is any molecule or compound except for the immunostimulatory nucleic acids described herein which can stimulate the humoral and/or cellular immune response. Non-nucleic acid adjuvants include, for instance, adjuvants that create a depot effect, immune stimulating adjuvants, adjuvants that create a depot effect and stimulate the immune system, and mucosal adjuvants.

An “adjuvant that creates a depot effect” as used herein is an adjuvant that causes an antigen to be slowly released in the body, thus prolonging the exposure of immune cells to the antigen. This class of adjuvants includes but is not limited to alum (e.g., aluminum hydroxide, aluminum phosphate); or emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720, AirLiquide, Paris, France); MF-59 (a squalene-in-water emulsion stabilized with Span 85 and Tween 80; Chiron Corporation, Emeryville, Calif.; and PROVAX (an oil-in-water emulsion containing a stabilizing detergent and a micelle-forming agent; IDEC, Pharmaceuticals Corporation, San Diego, Calif.).

An “immune stimulating adjuvant” is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21^{st} peak with HPLC fractionation; Aquila Biopharmaceuticals, Inc., Worcester, Mass.); poly[di(carboxylatophenoxy)phosphazene] (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); polyarginine (InterCell, Vienna, Austria); and Leishmania elongation factor (a purified Leishmania protein; Corixa Corporation, Seattle, Wash.).

“Adjuvants that create a depot effect and stimulate the immune system” are those compounds which have both of the above-identified functions. This class of adjuvants includes but is not limited to ISCOMs (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxpropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

A “non-nucleic acid mucosal adjuvant” as used herein is an adjuvant other than an immunostimulatory nucleic acid that is capable of inducing a mucosal immune response in a subject when administered to a mucosal surface in conjunction with an antigen. Mucosal adjuvants include but are not limited to bacterial toxins: e.g., Cholera toxin (CT), CT derivatives including but not limited to CT B subunit (CTB) (Wu et al. (1998) Vaccine 16:286-92, Tochikubo et al. (1998) Vaccine 16:150-5); CTD53 (Val to Asp) (Fontana et al. (1995) Infect Immun 63:2356-60); CTK97 (Val to Lys) (Fontana et al. (1995) Infect Immun 63:2356-60); CTK104 (Tyr to Lys) (Fontana et al. (1995) Infect Immun 63:2356-60); CTD53/K63 (Val to Asp, Ser to Lys) (Fontana et al. (1995) Infect Immun 63:2356-60); CTH54 (Arg to His) (Fontana et al. (1995) Infect Immun 63:2356-60); CTN107 (His to Asn) (Fontana et al. (1995) Infect Immun 63:2356-60); CTE114 (Ser to Glu) (Fontana et al. (1995) Infect Immun 63:2356-60); CTE112K (Glu to Lys) (Yamamoto et al. (1997) J Exp Med 185:1203-10); CTS61F (Serto Phe) (Yamamoto et al. (1997) J Exp Med 185:1203-10, Yamamoto et al. (1997) Proc Natl Acad Sci USA 94:5267-72); CTS106 (Pro to Ser) (Douce et al. (1997) Infect Immun 65:2821-8, Fontana et al. (1995) Infect Immun 63:2356-60); and CTK63 (Ser to Lys) (Douce et al. (1997) Infect Immun 65:2821-8, Fontana et al. (1995) Infect Immun 63:2356-60); zonula occludens toxin, zot, Escherichia coli heat-labile enterotoxin, Labile Toxin (LT), LT derivatives including but not limited to LT B subunit (LTB) (Verweij et al. (1998); LT7K (Arg to Lys) (Komase et al. (1998) Douce et al. (1995); LT61F (Ser to Phe) (Komase et al. (1998); LT112K (Glu to Lys) (Komase et al. (1998); LT118E (Gly to Glu) (Komase et al. (1998); LT146E (Arg to Glu) (Komase et al. (1998); LT192G (Arg to Gly) (Komase et al. (1998); LTK63 (Ser to Lys) (Marchetti et al. (1998) Douce et al. (1997 (1998) Di Tommaso et al. (1996); and LTR72 (Ala to Arg) (Giuliani et al. (1998) J Exp Med 187:1123-32); Pertussis toxin, P T (Lycke et al. (1992), Spangler B D (1992), Freytag and Clements (1999), Roberts et al. (1995) Wilson et al. (1995) including PT-9K/129G (Roberts et al. (1995) Cropley et al. (1995); toxin derivatives (see below) (Holmgren et al. (1993), Verweij et al. (1998), Rappuoli et al. (1995), Freytag and Clements (1999) Curr Top Microbiol Immunol 236:215-36); Lipid A derivatives (e.g., monophosphoryl lipid A, MPL) (Sasaki et al. (1998) Infect Immun 66:823-6, VanCott et al. (1998) J Immunol 160:2000-12); muramyl dipeptide (MDP) derivatives (Fukushima et al. (1996) Ogawa et al. (1989) Michalek et al. (1983) Morisaki et al. (1983); bacterial outer membrane proteins (e.g., outer surface protein A (OspA) lipoprotein of Borrelia burgdorferi, outer membrane protine of Neisseria meningitidis) (Marinaro et al. (1999) Van de Verg et al. (1996); oil-in-water emulsions (e.g., MF59) (Barchfield et al. (1999) Verschoor et al. (1999) O'Hagan (1998); aluminum salts (Isaka et al. (1998 (1999); and saponins (e.g., QS21, Aquila Biopharmaceuticals, Inc., Worcester, Mass.) (Sasaki et al. (1998) McNeal et al. (1998), ISCOMs, MF-59 (a squalene-in-water emulsion stabilized with Span 85 and Tween 80; Chiron Corporation, Emeryville, Calif.); the Seppic ISA series of Montanide adjuvants (e.g., Montanide ISA 720; AirLiquide, Paris, France); PROVAX (an oil-in-water emulsion containing a stabilizing detergent and a micelle-forming agent; IDEC Pharmaceuticals Corporation, San Diego, Calif.); Syntex Adjuvant Formulation (SAF; Syntex Chemicals, Inc., Boulder, Colo.); poly[di(carboxylatophenoxy)phosphazene] (PCPP polymer; Virus Research Institute, USA) and Leishmania elongation factor (Corixa Corporation, Seattle, Wash.).

Immune responses can also be induced or augmented by the co-administration or co-linear expression of cytokines (Bueler and Mulligan (1996); Chow et al. (1997); Geissler et al., (1997); Iwasaki et al. (1997); Kim et al. (1997)) or costimulatory molecules (Iwasaki et al. (1997); Tsuji et al. (1997)) with the immunostimulatory nucleic acids and anemia, thrombocytopenia, or neutropenia medicaments. The cytokines or costimulatory molecules can be administered directly with immunostimulatory nucleic acids or may be administered in the form of a nucleic acid vector that encodes the cytokine or costimulatory molecule, such that the cytokine or costimulatory molecule can be expressed in vivo. In one embodiment, the cytokine is administered in the form of a plasmid expression vector.

The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to IL-1, IL-2, IL-3, IL-4, IL-5, IL-6, IL-7, IL-10, IL-11, IL-12, IL-13, IL-15, IL-18, GM-CSF, G-CSF, IFN-γ, IFN-α, tumor necrosis factor (TNF), transforming growth factor (TGF)-α, Flt-3 ligand, and CD40 ligand. Cytokines play a role in directing the T cell response. Helper (CD4+) T cells and professional antigen presenting cells orchestrate the immune response of mammals through production of soluble factors that act on other immune system cells, including other T cells.

The term “effective amount” of an immunostimulatory nucleic acid and an anemia, thrombocytopenia, or neutropenia medicament refers to the amount necessary or sufficient to realize a desired biologic effect. For example, an effective amount of an immunostimulatory nucleic acid and an anemia medicament for treating or preventing anemia is that amount necessary to treat or prevent the development of anemia. Likewise, an effective amount of an immunostimulatory nucleic acid and a thrombocytopenia medicament for treating or preventing thrombocytopenia is that amount necessary to treat or prevent the development of thrombocytopenia. Combined with the teachings provided herein, by choosing among the various active compounds and weighing factors such as potency, relative bioavailability, subject body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular immunostimulatory nucleic acid or anemia, thrombocytopenia, or neutropenia medicament being administered (i.e., the type of nucleic acid, e.g., for a CpG nucleic acid, the number, context, and methylation status of CpG dinucleotides in the nucleic acid, the degree of modification of the oligonucleotide backbone), the size of the subject, and the severity of the disease or condition. One of ordinary skill in the art can empirically determine the effective amount of a particular immunostimulatory nucleic acid and/or anemia, thrombocytopenia, or neutropenia medicament and/or other therapeutic agent without undue experimentation. It should be noted that effective doses and routes of administration for many of the anemia, thrombocytopenia, and neutropenia medicaments, used alone, are well known in the art.

The immunostimulatory nucleic acid and anemia, thrombocytopenia, or neutropenia medicament are administered in a synergistic amount effective to treat or prevent anemia, thrombocytopenia, or neutropenia. A synergistic amount is that amount which produces a physiological response that is greater than the sum of the individual effects of the immunostimulatory nucleic acid and the anemia, thrombocytopenia, or neutropenia medicament alone. For instance, in some embodiments of the invention, the physiological effect is an increase in circulating platelets. A synergistic amount is that amount which produces an increase in circulating platelets that is greater than the sum of the increase in circulating platelets achieved by the separate administration of the same amounts of the immunostimulatory nucleic acid and the thrombocytopenia medicament alone. In other embodiments the physiological result is an increase in circulating erythrocytes. Yet other embodiments result in an increase in circulating neutrophils. In the case of thrombocytopenia medicaments, the physiologic response may be manifest as a reduction in bleeding and/or transfusion requirement. In the case of anemia medicaments, the physiologic response may be discernible as an improved exercise tolerance, reduced level of fatigue, and/or reduced transfusion requirement.

In some embodiments of the invention, the immunostimulatory nucleic acid is administered in an effective amount for preventing bacterial or viral infection. Immunostimulatory nucleic acids are known to be useful for preventing bacterial and viral infections. Bacterial and viral infections exacerbate and/or induce anemia, thrombocytopenia, or neutropenia. In this aspect of the invention, the immunostimulatory nucleic acid is administered to the subject in an amount effective to prevent bacterial and viral infection, and the anemia, thrombocytopenia, or neutropenia medicament is administered to the subject when symptoms or evidence of anemia, thrombocytopenia, or neutropenia appear. Thus, the immunostimulatory nucleic acid is administered to the subject and then the anemia, thrombocytopenia, or neutropenia medicament is subsequently administered to the subject or they are administered together at the same time. This method is particularly useful in subjects who are particularly susceptible to bacterial or viral disease, such as children, immunocompromised subjects, and elderly subjects.

For adult human subjects, doses of the immunostimulatory nucleic acid compounds described herein typically range from about 50 μg/dose to 20 mg/dose, more typically from about 80 μg/dose to 8 mg/dose, and most typically from about 800 μg/dose to 4 mg/dose. Stated in terms of subject body weight, typical dosages range from about 0.5 to 500 μg/kg/dose, more typically from about 1 to 100 μg/kg/dose, and most typically from about 10 to 50 μg/kg/dose. Doses will depend on factors including the route of administration, e.g., oral administration may require a substantially larger dose than subcutaneous administration.

In some instances, a sub-therapeutic dosage of the immunostimulatory nucleic acid and the anemia, thrombocytopenia, or neutropenia medicament are used. In some instances, the immunostimulatory nucleic acid and a sub-therapeutic dosage of the anemia, thrombocytopenia, or neutropenia medicament are used. It has been discovered, according to the invention, that when the two classes of drugs are used together, they can be administered in sub-therapeutic doses and still produce a desirable therapeutic result. A “sub-therapeutic dose” as used herein refers to a dosage which is less than that dosage which would produce a therapeutic result in the subject. Thus, the sub-therapeutic dose of an anemia, thrombocytopenia, or neutropenia medicament is one which would not produce the desired therapeutic result in the subject. Therapeutic doses of anemia, thrombocytopenia, or neutropenia medicaments are well known in the field of medicine for the treatment of anemia, thrombocytopenia, and neutropenia. These dosages have been extensively described in references such as Remington's Pharmaceutical Sciences, 18th ed. (1990), as well as many other medical references relied upon by the medical profession as guidance for the treatment of anemia, thrombocytopenia, and neutropenia. Therapeutic dosages of immunostimulatory nucleic acids have also been described in the art, and methods for identifying therapeutic dosages in subjects are described in more detail above.

In other aspects, the method of the invention involves administering a high dose of an anemia, thrombocytopenia, or neutropenia medicament to a subject, without inducing side effects. Ordinarily, when an anemia, thrombocytopenia, or neutropenia medicament is administered in a high dose, a variety of side effects can occur. (Discussed in more detail above, as well as in the medical literature). As a result of these side effects, the anemia, thrombocytopenia, or neutropenia medicament is not administered in such high doses, no matter what therapeutic benefits are derived. It was discovered, according to the invention, that such high doses of anemia, thrombocytopenia, or neutropenia medicaments which ordinarily induce side effects can be administered without inducing the side effects as long as the subject also receives an immunostimulatory nucleic acid. The type and extent of the side effects ordinarily induced by the anemia, thrombocytopenia, or neutropenia medicament will depend on the particular anemia, thrombocytopenia, or neutropenia medicament used.

In other embodiments of the invention, the immunostimulatory nucleic acid is administered on a routine schedule. The anemia, thrombocytopenia, or neutropenia medicament may also be administered on a routine schedule. Alternatively, the anemia, thrombocytopenia, or neutropenia medicament may be administered as symptoms arise. A “routine schedule” as used herein, refers to a predetermined designated period of time. The routine schedule may encompass periods of time which are identical or which differ in length, as long as the schedule is predetermined. For instance, the routine schedule may involve administration of the immunostimulatory nucleic acid on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the immunostimulatory nucleic acid on a daily basis for the first week, followed by a monthly basis for several months, and then every three months after that. Any particular combination would be covered by the routine schedule as long as it is determined ahead of time that the appropriate schedule involves administration on a certain day.

In other aspects, the invention relates to kits that are useful in the treatment of anemia, thrombocytopenia, and/or neutropenia. One kit of the invention includes a sustained-release vehicle containing an immunostimulatory nucleic acid and a container housing an anemia, thrombocytopenia, or neutropenia medicament and instructions for timing of administration of the immunostimulatory nucleic acid in the anemia, thrombocytopenia, or neutropenia medicament. A sustained-release vehicle is used herein in accordance with its prior art meaning of any device which slowly releases the immunostimulatory nucleic acid.

Such systems can avoid repeated administrations of the compounds, increasing convenience to the subject and the physician. Many types of sustained-release delivery systems are available and known to those of ordinary skill in the art. They include polymer-based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

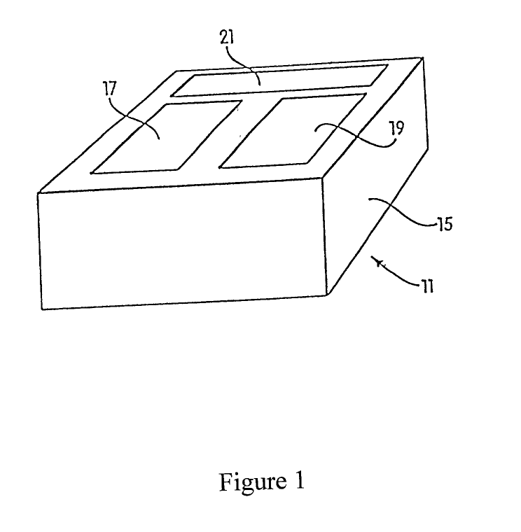

Referring to FIG. 1 depicting a kit 11, the anemia, thrombocytopenia, or neutropenia medicament 17 is housed in at least one container 19. The container 19 may be a single container housing all of the anemia, thrombocytopenia, or neutropenia medicament 17 together or it may be multiple containers or chambers housing individual dosages of the anemia, thrombocytopenia, or neutropenia medicament, such as a blister pack. The kit 11 also has instructions 21 for timing of administration of the anemia, thrombocytopenia, or neutropenia medicament, plus a box-like package 15. The instructions 21 would direct the subject having anemia, thrombocytopenia, or neutropenia or at risk of developing anemia, thrombocytopenia, or neutropenia to take the anemia, thrombocytopenia, or neutropenia medicament at the appropriate time. For instance, the appropriate time for delivery of the medicament may be as the symptoms occur. Alternatively, the appropriate time for administration of the medicament may be on a routine schedule such as weekly, monthly, or yearly.

Another kit 11 of the invention includes at least one container 19 housing an immunostimulatory nucleic acid and at least one container housing an anemia, thrombocytopenia, or neutropenia medicament 17 and instructions 21 for administering the compositions in effective amounts for inducing a synergistic immune response in the subject. The immunostimulatory nucleic acid and anemia, thrombocytopenia, or neutropenia medicament may be housed in single containers or in separate compartments or containers, such as single dose compartments. The instructions in the kit direct the subject to take the immunostimulatory nucleic acid and the anemia, thrombocytopenia, or neutropenia medicament in amounts which will produce a synergistic immune response. The immunostimulatory nucleic acid and the anemia, thrombocytopenia, or neutropenia medicament may be administered simultaneously or separately, as long as they are administered close enough in time to produce a synergistic response.

In other aspects of the invention, a pharmaceutical composition is provided. The pharmaceutical composition includes an immunostimulatory nucleic and an anemia, thrombocytopenia, or neutropenia medicament formulated in a pharmaceutically acceptable carrier and present in the pharmaceutical composition in an effective amount for preventing or treating anemia, thrombocytopenia, or neutropenia. The effective amount for preventing or treating anemia, thrombocytopenia, or neutropenia is that amount which completely or partially prevents the development of, prevents the worsening of, or treats the established existence of, anemia, thrombocytopenia, or neutropenia. In some instances, the effective amount for preventing or treating anemia, thrombocytopenia, or neutropenia completely or partially prevents or treats clinical symptoms of anemia, thrombocytopenia, or neutropenia.

Certain in vitro assays may be useful in determining a therapeutically effective amount of a particular nucleic acid. The relative effective amount of immunostimulatory nucleic acid useful for inducing an immune response can be assessed using the in vitro assays with respect to stimulation index in comparison to known immunostimulatory nucleic acids. The stimulation index can be used to determine an effective amount of the particular oligonucleotide for the particular subject, and the dosage can be adjusted upwards or downwards to achieve the desired levels in the subject. Therapeutically effective amounts can also be determined from animal models. A therapeutically effective dose can also be determined from human data for immunostimulatory nucleic acids which have been tested in humans (human clinical trials have been initiated) and for compounds which are known to exhibit similar pharmacological activities, such as other adjuvants, e.g., LT and other antigens for vaccination purposes. The applied dose can be adjusted based on the relative bioavailability and potency of the administered compound. Adjusting the dose to achieve maximal efficacy based on the methods described above and other methods as are well known in the art is well within the capabilities of the ordinarily skilled artisan. Anemia, thrombocytopenia, and neutropenia medicaments are well known in the art. The amounts of anemia, thrombocytopenia, and neutropenia medicaments can be adjusted when they are combined with immunostimulatory nucleic acids by routine experimentation.

In addition to clinical outcomes measured in terms of physiology, in vitro assays measuring erythrocyte, platelet, and granulocyte counts may be used in determining a therapeutically effective amount of a particular nucleic acid. These methods are standard medical laboratory techniques that are well known in the art. In common practice such measurements may be be made by automated cell counting devices designed for that purpose, or they may be performed manually. Manual counts may be more accurate than automated counts when cell counts are particularly low.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Anemia, thrombocytopenia, or neutropenia medicaments can be administered by any effective route for administering such medications. Preferably, they are injected locally, ingested, or administered by systemic routes. Systemic routes include enteral (e.g., oral) and parenteral (e.g., intravenous).

For use in therapy, an effective amount of the immunostimulatory nucleic acid can be administered to a subject by any mode that delivers the nucleic acid to the desired site, e.g., mucosal, systemic. “Administering” the immunostimulatory nucleic acid of the present invention may be accomplished by any means known to the skilled artisan. Preferred routes of administration include but are not limited to enteral, oral, parenteral, intramuscular, intravenous, subcutaneous, transdermal, intranasal, intratracheal, inhalation, ocular, vaginal, and rectal.

For oral administration, the compounds (i.e., immunostimulatory nucleic acids, anemia, thrombocytopenia, or neutropenia medicament, other therapeutic agent) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents may be added, such as the cross-linked PVP, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, PVP, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoro-ethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic, such as the immunostimulatory capacity of the nucleic acids (see, for example, Sciarra and Cutie, “Aerosols,” in Remington's Pharmaceutical Sciences, 18th edition (1990) pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. It has been found according to the invention that subcutaneous administration of the immunostimulatory nucleic acid results in a systemic effect. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, ointments, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533 (1990) which is incorporated herein by reference.

The immunostimulatory nucleic acids and anemia, thrombocytopenia, or neutropenia medicament may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: acetic, benzene sulphonic, citric, formic, hydrobromic, hydrochloric, maleic, malonic, methane sulphonic, naphthalene-2-sulphonic, nitric, phosphoric, p-toluene sulphonic, salicylic, succinic, sulphuric, and tartaric. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v). The pharmaceutical compositions of the invention contain an effective amount of an immunostimulatory nucleic acid and optionally anemia, thrombocytopenia, or neutropenia medicament and/or other therapeutic agents optionally included in a pharmaceutically acceptable carrier. The term “pharmaceutically acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method for treating or preventing anemia, comprising:
administering to a subject having anemia or at risk of developing anemia a combination of an immunostimulatory nucleic acid and an anemia medicament in an effective amount to treat or prevent the anemia.

2. The method of claim 1, wherein the immunostimulatory nucleic acid is a CpG nucleic acid and wherein the combination is a synergistic combination.

3. The method of claim 1, wherein the immunostimulatory nucleic acid is a methylated CpG nucleic acid.

4. The method of claim 1, wherein the immunostimulatory nucleic acid is a T-rich nucleic acid.

5. The method of claim 1, wherein the immunostimulatory nucleic acid is a poly-G nucleic acid.

6. The method of claim 1, wherein the immunostimulatory nucleic acid is any combination of nucleic acids selected from the group consisting of CpG nucleic acids, methylated CpG nucleic acids, T-rich nucleic acids, and poly-G nucleic acids.

7. The method of claim 1, wherein the anemia medicament is a medicament selected from the group consisting of recombinant erythropoietin (EPO), recombinant granulocyte-macrophage colony-stimulating factor (GM-CSF), recombinant granulocyte colony-stimulating factor (G-CSF), recombinant interleukin 11 (IL-11), ferrous iron, ferric iron, vitamin B12, vitamin B6, vitamin C, vitamin D, calcitriol, alphacalcidol, folate, androgen, and carnitine.

8. The method of claim 1, wherein the anemia medicament is recombinant EPO.

9. The method of claim 1, wherein the immunostimulatory nucleic acid is administered concurrently with the anemia medicament.

10. The method of claim 1, wherein the immunostimulatory nucleic acid has a modified backbone.

11. The method of claim 10, wherein the modified backbone comprises a phosphate backbone modification.

12. The method of claim 10, wherein the modified backbone is a phosphorothioate backbone.

13. The method of claim 1, wherein the subject is preparing to undergo chemotherapy.

14. The method of claim 1, wherein the subject is preparing to undergo radiation treatment.

15. The method of claim 1, wherein the subject has received at least one dose of chemotherapy.

16. The method of claim 1, wherein the subject has received at least one radiation treatment.

17. The method of claim 1, wherein the immunostimulatory nucleic acid is administered systemically and the anemia medicament is administered locally.

18. The method of claim 1, wherein the immunostimulatory nucleic acid comprises a sequence selected from the group consisting of SEQ ID NO:1- SEQ ID NO:133.

19. The method of claim 1, wherein the immunostimulatory nucleic acid is administered on a variable schedule.

20. The method of claim 1, wherein the immunostimulatory nucleic acid is administered on a routine schedule.

21. The method of claim 1, wherein the immunostimulatory nucleic acid is administered in a sustained-release vehicle.

22. The method of claim 20, wherein the immunostimulatory nucleic acid is administered on a routine schedule selected from the group consisting of every day, at least twice a week, at least three times a week, at least four times a week, at least five times a week, at least six times a week, every week, every other week, every third week, every fourth week, every month, every two months, every three months, every four months, and every six months.

23. A method for decreasing the dose of an anemia medicament, comprising:
administering to a subject having anemia or at risk of developing anemia an anemia medicament in a sub-therapeutic dosage and an immunostimulatory nucleic acid, wherein the anemia medicament in a sub-therapeutic dosage and the immunostimulatory nucleic acid are effective to treat or prevent anemia in the subject.

24. The method of claim 23, wherein the immunostimulatory nucleic acid is a CpG nucleic acid.

25. The method of claim 23, wherein the immunostimulatory nucleic acid is a methylated CpG nucleic acid.

26. The method of claim 23, wherein the immunostimulatory nucleic acid is a T-rich nucleic acid.

27. The method of claim 23, wherein the immunostimulatory nucleic acid is a poly-G nucleic acid.

28. The method of claim 23, wherein the immunostimulatory nucleic acid is any combination of nucleic acids selected from the group consisting of CpG nucleic acids, methylated CpG nucleic acids, T-rich nucleic acids, and poly-G nucleic acids.

29. The method of claim 23, wherein the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine.

30. The method of claim 23, wherein the anemia medicament is recombinant EPO.

31. The method of claim 23, wherein the immunostimulatory nucleic acid has a modified backbone.

32. The method of claim 31, wherein the modified backbone comprises a phosphate backbone modification.

33. The method of claim 31, wherein the modified backbone is a phosphorothioate backbone.

34. A method for treating or preventing anemia, comprising:
administering to a subject having anemia or at risk of developing anemia an immunostimulatory nucleic acid selected from the group consisting of a methylated CpG nucleic acid, a T-rich nucleic acid, a poly-G nucleic acid, a nucleic acid having a phosphorothioate backbone, and any combination thereof, wherein the nucleic acid having a phosphorothioate backbone is not a CpG nucleic acid, in an effective amount to treat or prevent the anemia.

35. A method for treating or preventing thrombocytopenia, comprising:
administering to a subject having thrombocytopenia or at risk of developing thrombocytopenia a combination of an immunostimulatory nucleic acid and a thrombocytopenia medicament in an effective amount to treat or prevent the thrombocytopenia.

36. The method of claim 35, wherein the immunostimulatory nucleic acid is a CpG nucleic acid and wherein the combination is a synergistic combination.

37. The method of claim 35, wherein the immunostimulatory nucleic acid is a methylated CpG nucleic acid.

38. The method of claim 35, wherein the immunostimulatory nucleic acid is a T-rich nucleic acid.

39. The method of claim 35, wherein the immunostimulatory nucleic acid is a poly-G nucleic acid.

40. The method of claim 35, wherein the immunostimulatory nucleic acid is any combination of nucleic acids selected from the group consisting of CpG nucleic acids, methylated CpG nucleic acids, T-rich nucleic acids, and poly-G nucleic acids.

41. The method of claim 35, wherein the thrombocytopenia medicament is a medicament selected from the group consisting of a glucocorticoid, recombinant thrombopoietin (TPO), recombinant megakaryocyte growth and development factor (MGDF), pegylated recombinant MGDF, lisophylline, recombinant IL-1, recombinant IL-3, recombinant IL-6, and recombinant IL-11.

42. The method of claim 35, wherein the thrombocytopenia medicament is recombinant TPO.

43. The method of claim 35, wherein the immunostimulatory nucleic acid is administered concurrently with the thrombocytopenia medicament.

44. The method of claim 35, wherein the immunostimulatory nucleic acid has a modified backbone.

45. The method of claim 44, wherein the modified backbone comprises a phosphate backbone modification.

46. The method of claim 44, wherein the modified backbone is a phosphorothioate backbone.

47. The method of claim 35, wherein the subject is preparing to undergo chemotherapy.

48. The method of claim 35, wherein the subject is preparing to undergo radiation treatment.

49. The method of claim 35, wherein the subject has received at least one dose of chemotherapy.

50. The method of claim 35, wherein the subject has received at least one radiation treatment.

51. The method of claim 35, wherein the immunostimulatory nucleic acid is administered systemically and the thrombocytopenia medicament is administered locally.

52. The method of claim 35, wherein the immunostimulatory nucleic acid comprises a sequence selected from the group consisting of SEQ ID NO:1-SEQ ID NO:133.

53. The method of claim 35, wherein the immunostimulatory nucleic acid is administered on a variable schedule.

54. The method of claim 35, wherein the immunostimulatory nucleic acid is administered on a routine schedule.

55. The method of claim 35, wherein the immunostimulatory nucleic acid is administered in a sustained-release vehicle.

56. The method of claim 54, wherein the immunostimulatory nucleic acid is administered on a routine schedule selected from the group consisting of every day, at least twice a week, at least three times a week, at least four times a week, at least five times a week, at least six times a week, every week, every other week, every third week, every fourth week, every month, every two months, every three months, every four months, and every six months.

57. A method for decreasing the dose of a thrombocytopenia medicament, comprising:
administering to a subject having thrombocytopenia or at risk of developing thrombocytopenia a thrombocytopenia medicament in a sub-therapeutic dosage and an immunostimulatory nucleic acid, wherein the thrombocytopenia medicament in a sub-therapeutic dosage and the immunostimulatory nucleic acid are effective to treat or prevent thrombocytopenia in the subject.

58. The method of claim 57, wherein the immunostimulatory nucleic acid is a CpG nucleic acid.

59. The method of claim 57, wherein the immunostimulatory nucleic acid is a methylated CpG nucleic acid.

60. The method of claim 57, wherein the immunostimulatory nucleic acid is a T-rich nucleic acid.

61. The method of claim 57, wherein the immunostimulatory nucleic acid is a poly-G nucleic acid.

62. The method of claim 57, wherein the immunostimulatory nucleic acid is any combination of nucleic acids selected from the group consisting of CpG nucleic acids, methylated CpG nucleic acids, T-rich nucleic acids, and poly-G nucleic acids.

63. The method of claim 57, wherein the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, recombinant IL-11, and recombinant G-CSF.

64. The method of claim 57, wherein the thrombocytopenia medicament is a glucocorticoid.

65. The method of claim 57, wherein the thrombocytopenia medicament is recombinant TPO.

66. The method of claim 57, wherein the thrombocytopenia medicament comprises recombinant MGDF.

67. The method of claim 57, wherein the immunostimulatory nucleic acid has a modified backbone.

68. The method of claim 67, wherein the modified backbone comprises a phosphate backbone modification.

69. The method of claim 67, wherein the modified backbone is a phosphorothioate backbone.

70. A method for treating or preventing thrombocytopenia, comprising:
administering to a subject having thrombocytopenia or at risk of developing thrombocytopenia an immunostimulatory nucleic acid selected from the group consisting of a methylated CpG nucleic acid, a T-rich nucleic acid, a poly-G nucleic acid, a nucleic acid having a phosphorothioate backbone, and any combination thereof, wherein the nucleic acid having a phosphorothioate backbone is not a CpG nucleic acid, in an effective amount to treat or prevent the thrombocytopenia.

71. A method for treating or preventing neutropenia, comprising:
administering to a subject having neutropenia or at risk of developing neutropenia a combination of an immunostimulatory nucleic acid and a neutropenia medicament in an effective amount to treat or prevent the neutropenia.

72. The method of claim 71, wherein the immunostimulatory nucleic acid is a CpG nucleic acid and wherein the combination is a synergistic combination.

73. The method of claim 71, wherein the immunostimulatory nucleic acid is a methylated CpG nucleic acid.

74. The method of claim 71, wherein the immunostimulatory nucleic acid is a T-rich nucleic acid.

75. The method of claim 71, wherein the immunostimulatory nucleic acid is a poly-G nucleic acid.

76. The method of claim 71, wherein the immunostimulatory nucleic acid is any combination of nucleic acids selected from the group consisting of CpG nucleic acids, methylated CpG nucleic acids, T-rich nucleic acids, and poly-G nucleic acids.

77. The method of claim 71, wherein the neutropenia medicament is a medicament selected from the group consisting of glucocorticoid, recombinant G-CSF, recombinant GM-CSF, recombinant macrophage colony-simulating factor (M-CSF), recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin.

78. The method of claim 71, wherein the neutropenia medicament is recombinant G-CSF.

79. The method of claim 71, wherein the immunostimulatory nucleic acid is administered concurrently with the neutropenia medicament.

80. The method of claim 71, wherein the immunostimulatory nucleic acid has a modified backbone.

81. The method of claim 80, wherein the modified backbone comprises a phosphate backbone modification.

82. The method of claim 80, wherein the modified backbone is a phosphorothioate backbone.

83. The method of claim 71, wherein the subject is immunocompromised or at risk of becoming immunocompromised.

84. The method of claim 71, wherein the subject is preparing to undergo chemotherapy.

85. The method of claim 71, wherein the subject is preparing to undergo radiation treatment.

86. The method of claim 71, wherein the subject has received at least one dose of chemotherapy.

87. The method of claim 71, wherein the subject has received at least one radiation treatment.

88. The method of claim 71, wherein the immunostimulatory nucleic acid is administered systemically and the neutropenia medicament is administered locally.

89. The method of claim 71, wherein the immunostimulatory nucleic acid comprises a sequence selected from the group consisting of SEQ ID NO:1-SEQ ID NO:133.

90. The method of claim 71, wherein the immunostimulatory nucleic acid is administered on a variable schedule.

91. The method of claim 71, wherein the immunostimulatory nucleic acid is administered on a routine schedule.

92. The method of claim 71, wherein the immunostimulatory nucleic acid is administered in a sustained-release vehicle.

93. The method of claim 91, wherein the immunostimulatory nucleic acid is administered on a routine schedule selected from the group consisting of every day, at least twice a week, at least three times a week, at least four times a week, at least five times a week, at least six times a week, every week, every other week, every third week, every fourth week, every month, every two months, every three months, every four months, and every six months.

94. A method for increasing the dose of a neutropenia medicament, comprising:
administering to a subject having neutropenia or at risk of developing neutropenia a medicament in a dose which ordinarily induces side effects, and
administering to the subject an effective amount for preventing the induction of side effects by the neutropenia medicament of an immunostimulatory nucleic acid.

95. The method of claim 94, wherein the immunostimulatory nucleic acid is any combination of nucleic acids selected from the group consisting of CpG nucleic acids, methylated CpG nucleic acids, T-rich nucleic acids, and poly-G nucleic acids.

96. The method of claim 94, wherein the immunostimulatory nucleic acid is a CpG nucleic acid.

97. The method of claim 94, wherein the neutropenia medicament is a medicament selected from the group consisting of glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin.

98. The method of claim 94, wherein the neutropenia medicament is recombinant G-CSF.

99. The method of claim 94, wherein the immunostimulatory nucleic acid has a modified backbone.

100. The method of claim 99, wherein the modified backbone comprises a phosphate backbone modification.

101. The method of claim 100, wherein the modified backbone is a phosphorothioate backbone.

102. A method for decreasing the dose of a neutropenia medicament, comprising:
administering to a subject having neutropenia or at risk of developing neutropenia a a neutropenia medicament in a sub-therapeutic dosage and an immunostimulatory nucleic acid, wherein the neutropenia medicament in a sub-therapeutic dosage and the immunostimulatory nucleic acid produce a therapeutic are effective to treat or prevent neutropenia in the subject.

103. The method of claim 102, wherein the immunostimulatory nucleic acid is a CpG nucleic acid.

104. The method of claim 102, wherein the immunostimulatory nucleic acid is a methylated CpG nucleic acid.

105. The method of claim 102, wherein the immunostimulatory nucleic acid is a T-rich nucleic acid.

106. The method of claim 102, wherein the immunostimulatory nucleic acid is a poly-G nucleic acid.

107. The method of claim 102, wherein the immunostimulatory nucleic acid is any combination of nucleic acids selected from the group consisting of CpG nucleic acids, methylated CpG nucleic acids, T-rich nucleic acids, and poly-G nucleic acids.

108. The method of claim 102, wherein the neutropenia medicament is selected from the group consisting of glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin.

109. The method of claim 102, wherein the neutropenia medicament is recombinant G-CSF.

110. The method of claim 102, wherein the immunostimulatory nucleic acid has a modified backbone.

111. The method of claim 110, wherein the modified backbone comprises a phosphate backbone modification.

112. The method of claim 110, wherein the modified backbone is a phosphorothioate backbone.

113. A method for treating or preventing neutropenia, comprising:
administering to a subject having neutropenia or at risk of developing neutropenia an immunostimulatory nucleic acid selected from the group consisting of a methylated CpG nucleic acid, a T-rich nucleic acid, a poly-G nucleic acid, a nucleic acid having a phosphorothioate backbone, and any combination thereof, wherein the nucleic acid having a phosphorothioate backbone is not a CpG nucleic acid, in an effective amount to treat or prevent the neutropenia.

114. A pharmaceutical composition, comprising:
an immunostimulatory nucleic acid and an anemia medicament, formulated in a pharmaceutically acceptable carrier and in an effective amount for treating or preventing anemia.

115. The pharmaceutical composition of claim 114, wherein the immunostimulatory nucleic acid is any combination of nucleic acids selected from the group consisting of CpG nucleic acids, methylated CpG nucleic acids, T-rich nucleic acids, and poly-G nucleic acids.

116. The pharmaceutical composition of claim 114, wherein the immunostimulatory nucleic acid is a CpG nucleic acid.

117. The pharmaceutical composition of claim 114, wherein the immunostimulatory nucleic acid has a modified backbone.

118. The pharmaceutical composition of claim 117, wherein the modified backbone comprises a phosphate backbone modification.

119. The pharmaceutical composition of claim 117, wherein the modified backbone is a phosphorothioate backbone.

120. The pharmaceutical composition of claim 114, wherein the anemia medicament is selected from the group consisting of recombinant EPO, recombinant GM-CSF, recombinant G-CSF, recombinant IL-11, ferrous iron, ferric iron, vitamin B12, vitamin B6, vitamin C, vitamin D, calcitriol, alphacalcidol, folate, androgen, and carnitine.

121. The pharmaceutical composition of claim 114, wherein the anemia medicament is recombinant EPO.

122. A pharmaceutical composition, comprising:
an immunostimulatory nucleic acid and a thrombocytopenia medicament, formulated in a pharmaceutically acceptable carrier and in an effective amount for treating or preventing thrombocytopenia.

123. The pharmaceutical composition of claim 122, wherein the immunostimulatory nucleic acid is any combination of nucleic acids selected from the group consisting of CpG nucleic acids, methylated CpG nucleic acids, T-rich nucleic acids, and poly-G nucleic acids.

124. The pharmaceutical composition of claim 122, wherein the immunostimulatory nucleic acid is a CpG nucleic acid.

125. The pharmaceutical composition of claim 122, wherein the immunostimulatory nucleic acid has a modified backbone.

126. The pharmaceutical composition of claim 125, wherein the modified backbone comprises a phosphate backbone modification.

127. The pharmaceutical composition of claim 125, wherein the modified backbone is a phosphorothioate backbone.

128. The pharmaceutical composition of claim 122, wherein the thrombocytopenia medicament is selected from the group consisting of a glucocorticoid, recombinant TPO, recombinant MGDF, pegylated recombinant MGDF, lisophylline, recombinant IL-1, recombinant IL-3, recombinant IL-6, recombinant IL-11, and recombinant G-CSF.

129. The pharmaceutical composition of claim 122, wherein the thrombocytopenia medicament is recombinant TPO.

130. A pharmaceutical composition, comprising:
an immunostimulatory nucleic acid and a neutropenia medicament, formulated in a pharmaceutically acceptable carrier and in an effective amount for treating or preventing neutropenia.

131. The pharmaceutical composition of claim 130, wherein the immunostimulatory nucleic acid is any combination of nucleic acids selected from the group consisting of CpG nucleic acids, methylated CpG nucleic acids, T-rich nucleic acids, and poly-G nucleic acids.

132. The pharmaceutical composition of claim 130, wherein the immunostimulatory nucleic acid is a CpG nucleic acid.

133. The pharmaceutical composition of claim 130, wherein the immunostimulatory nucleic acid has a modified backbone.

134. The pharmaceutical composition of claim 133, wherein the modified backbone comprises a phosphate backbone modification.

135. The pharmaceutical composition of claim 133, wherein the modified backbone is a phosphorothioate backbone.

136. The pharmaceutical composition of claim 130, wherein the neutropenia medicament is selected from the group consisting of glucocorticoid, recombinant G-CSF, recombinant GM-CSF, recombinant M-CSF, recombinant IL-1, recombinant IL-3, recombinant IL-6, immunoglobulin, androgens, recombinant IFN-γ, small molecule G-CSF mimetics, G-CSF receptor antagonists, IL-3 receptor antagonists, and uteroferrin.

137. The pharmaceutical composition of claim 130, wherein the neutropenia medicament is recombinant G-CSF.
